(19) United States
(12) Patent Application Publication
(10) Pub. No.: US 2023/0218831 A1
(43) Pub. Date: Jul. 13, 2023

(54) Electronic system for a drug delivery device and drug delivery device

(71) Applicant: Sanofi, Paris (FR)

(72) Inventors: Paul Richard Draper, Warwick Warwickshire (GB); Anthony Paul Morris, Warwick Warwickshire (GB); Ronald Antony Smith, Warwick Warwickshire (GB); Philipp Troebner, Frankfurt am Main (DE)

(21) Appl. No.: 17/928,137

(22) PCT Filed: Mar. 25, 2021
(86) PCT No.: PCT/EP2021/057671

§ 371 Date: Nov. 28, 2022

(30) Foreign Application Priority Data
Jun. 18, 2020 (EP) EP20315305.1

(51) Int. Cl. A61M 5/315

(52) U.S. Cl. CPC A61M 5/31568; A61M 5/3155; A61M 5/3156; A61M 5/31535; A61M 2205/52

(57) ABSTRACT

An electronic system for a drug delivery device is provided. The system includes an electronic control unit configured to control an operation of the system, and an electrical motion sensing unit that is configured to generate one or more data signals. The data signals are suitable to quantify relative rotational movement between a first member and a second member during a dose setting operation for setting a dose to be delivered by the drug delivery device and/or a dose delivery operation for delivering the set dose. The system is configured to perform a comparison operation to compare first data to second data. The first data indicating a first relative angular position of the first member and the second member, and being derivable from one or more data signals. The second data indicating a second relative angular position of the first member and the second member.

### CROSS-REFERENCE TO RELATED APPLICATIONS

The present application is the national stage entry of International Patent Application No. PCT/EP2021/057671, filed on Mar. 25, 2021, and claims priority to Application No. EP 20315305.1, filed on Jun. 18, 2020, the disclosures of which are incorporated herein by reference.

### TECHNICAL FIELD

The present disclosure relates to an electronic system for a drug delivery device. The present disclosure further relates to a drug delivery device, which preferably comprises the electronic system.

### BACKGROUND

Drug delivery devices using electronics are becoming increasingly popular in the pharmaceutical industry as well as for users or patients. However, especially if the device is designed to be self-contained, that is to say without a connector for a connection to an external electrical power source which is necessary to provide electrical power for the operation of the device, the management of the resources of a power supply integrated into the device is particularly important. Moreover, it is, of course, important that the electronic system functions as reliable as possible, e.g. when calculating or providing information related to a dose having been set or having been dispensed from the device.

### SUMMARY

This disclosure provides improvements for drug delivery devices comprising an electronic system or electronic systems for drug delivery devices.

One aspect of the disclosure relates to an electronic system for a drug delivery device. Another aspect of the disclosure relates to a drug delivery device comprising the electronic system. Accordingly, the features described herein with relation to the drug delivery device should be considered as being disclosed for the electronic system and vice versa. Likewise, features disclosed in conjunction with different embodiments can be combined with one another.

In one embodiment, the electronic system for the drug delivery device comprises an electronic control unit. The electronic control unit may be configured to control an operation of the electronic system. The electronic control unit may be or may comprise an electronic processor, such as a microcontroller or an ASIC, for example. The electronic system further comprises an electrical motion sensing unit. The electrical motion sensing unit, when operated, may be configured to generate one or more (data) signals, e.g. electrical signals. The data signal(s) may be suitable to quantify relative rotational movement between a first member and a second member, especially during a dose setting operation for setting a dose to be delivered by the drug delivery device, e.g. a dose of drug, and/or a dose delivery operation for delivering the set dose. The motion sensing unit may comprise one or more sensors and/or one or more emitters, e.g. one or more optoelectronic radiation sensors and/or one or more optoelectronic radiation emitters. The second member and/or the first member may be part of the drug delivery device and/or the electronic system. The electronic system, particularly the motion sensing unit, may be configured to generate data signal(s) in response to movement of the first member relative to the second member.

In one embodiment, the electronic system is configured to perform a comparison operation. The comparison operation may be performed by the electronic control unit or an electronic comparison unit which is separate from the electronic control unit. The comparison operation may be provided and/or configured to compare first data and second data. The first data and/or the second data may comprise data on one or more relative angular positions, preferably angular positions of the first member relative to the second member. The axis relative to which the angular positions are determined may be the rotation axis of the relative rotation between the first member and the second member. Based on the result of the comparison, the electronic system may be configured to trigger different operations or adjust one or more operations of the electronic system. The first data is preferably data indicative for a first relative angular position of the first member and the second member. The first data is expediently derivable or derived from one or more data signals generatable or generated by the motion sensing unit, particularly when the motion sensing unit operates during the dose setting operation or the dose delivery operation, expediently during that operation which should be sensed by the sensing unit. The second data, preferably, is or comprises data indicative for a second relative angular position of the first member and the second member. The result of the comparison of the first data in the second data may be that the first angular position and the second angular position are equal (if applicable two different angular positions arranged at an angular distance which is within a tolerance limit set for the comparison are considered as equal) or different (if applicable two different angular positions outside of a tolerance limit set for the comparison are considered as different). The tolerance limit may be less than or equal to one unit setting increment and/or less than or equal to a predefined angle, e.g. less than or equal to one of the following angles: 25°, 20°, 15° and or greater than or equal to one of the following angles: 5°, 10°, 15°. The unit setting increment will be explained in more detail further below and may be the minimum angle of rotation which is required to set a dose in the dose setting operation. The difference in the relative position, if detected during the comparison, may be used to adjust dose information derived from relative rotational movement between the first member and the second member during the relevant operation, i.e. dose setting operation or dose delivery operation, which is sensed and/or evaluated via the motion sensing unit. Expediently, the comparison operation is performed before the final dose information on the operation which is monitored via the motion sensing unit is transferred from the electronic control unit to another entity, such as for dose logging purposes. The dose information may be calculated by the electronic control unit or a dedicated calculation unit.

The comparison operation may increase the user safety and improve the accuracy of dose information determination in the drug delivery device or the electronic system. For example, it can be checked whether the first relative angular position is the one which is expected by the system, e.g. the one indicated by the second data and, if the check is positive, it can be assumed that the device has not been tampered with or functions properly. If the check is negative, appropriate corrective measures can be taken or an alarm can be triggered.

In one embodiment, the electronic system is configured to quantify the relative rotational movement of the first member and the second member in whole-number multiples of one unit setting increment or whole-number multiples of the predefined angle. The unit setting increment may be an angle by which a dose member can be or has to be rotated during a dose setting operation in order to set a dose. The dose member may be part of a dose setting and drive mechanism of the electronic system or the device. A rotation of the dose member from one stable position to the adjacent stable position may involve a rotation by an angle of one unit setting increment. Two adjacent stable positions may be separated by one unit setting increment. The respective stable position may be defined by a ratchet interface.

In one embodiment, the first data is derivable or derived from the first data signal, preferably from the very first data signal, generatable or generated by the electrical motion sensing unit, particularly after the motion sensing unit was switched on or became operative.

In one embodiment, the first data is data indicative for the first detectable relative angular position between the first member and the second member, which can be sensed or detected via the motion sensing unit during the respective operation, e.g. during the dose delivery operation or the dose setting operation.

In one embodiment, the second data is storable or is stored in the electronic system, preferably before the first data is derived or derivable from the first data signal. In other words, the second data may be retrievable from the electronic system. Preferably, the second data is not a data which has to be generated during the operation which is being monitored or sensed by the motion sensing unit. If the operation which is being monitored or sensed by the motion sensing unit is a dose delivery operation, the second data may be data generated during a previous operation, e.g. a previous dose setting operation or a previous dose delivery operation. The dose of the previous dose operation, preferably, has been dispensed already before the first data is generated.

In one embodiment, the second data is or comprises data indicative for the relative angular position between the first member and the second member at the end of a previous dose setting or dose delivery operation and/or before commencement of the dose setting operation or the dose delivery operation. Especially, the second data may be stored at the end of the previous operation before the electrical power consumption of the electronic system is reduced or the system is switched off.

In one embodiment, the electronic system comprises a, preferably non-transitory, memory. The second data on the second relative angular position may be storable, e.g. reliably, in the memory. Thus, even if the power consumption of the electronic system is reduced or the electronic system is switched off, this information may still be available for subsequent operations. In other words, the memory may be used to cache positional information or data on the relative angular position of the first member and the second member after completion of one operation. This information may then be compared with the relative position detected by the motion sensing unit at the onset of a subsequent operation. At the end of the subsequent operation, the data indicative for the relative position after that operation may be stored in the memory again for the next operation. The types of operation for which the positions are compared are expediently identical. That is to say if the second data is stored during or at the end of a dose delivery operation, then the subsequent operation which is sensed via the motion sensing unit and for or during which the comparison operation is performed is also a dose delivery operation. The same holds for the dose setting operation. The size of the set or delivered dose may, of course, vary between subsequent operations of the same type, i.e. setting operation or delivery operation. The system may be configured to store positional data indicative for an end position of the first and second member after an operation has been completed in the memory.

In one embodiment, the second data is derived or derivable from the one or more data signals generated by the electrical motion sensing unit during or at the end of a previous dose delivery operation. At the end of the previous dose delivery operation this data may be stored in the memory.

In one embodiment, the memory for storing the second data is provided in a unit different from the electronic control unit. The memory may be provided in a clock or timer unit, e.g. in a real-time clock of the electronic system.

In an embodiment, the second data for the comparison operation is retrievable or retrieved from the memory, e.g. the memory of the real-time clock.

In one embodiment, the electrical motion sensing unit is operative, preferably only, during the dose delivery operation or the dose setting operation. That is to say only one of these operations may be monitored or sensed by the motion sensing unit. In other words, the electronic system may be configured such that only relative movement during the dose setting operation or the dose delivery operation, preferably only during the dose delivery operation, is quantified. Gathering dose information on the delivered dose, e.g. on the size of the delivered dose which may be linked to the rotation in unit setting increments during the dose delivery operation, by monitoring the dose delivery operation is particularly advantageous as this is what matters most for the patient or user as only when performing the dose delivery operation it can be ensured that drug actually is delivered from a device.

In one embodiment, the electronic system is configured, such that the motion sensing unit is rendered operative, e.g. switched on, after, preferably only after, the dose setting operation or the dose delivery operation has been commenced. This may be achieved by an appropriate use signal being generated only after relative rotation has begun. Motion sensing units often have a comparatively high power consumption and, therefore, it is advantageous to reduce the time in which the motion sensing unit can drain power from an electrical power supply of the electronic system as far as possible, expediently without affecting the functionality of the system. Activating the unit or rendering the motion sensing unit operative only after the respective operation which should be sensed by the motion sensing unit has commenced ensures that the motion sensing unit is indeed required to monitor this operation as it is certain that the user has decided to perform the operation, e.g. by moving a user interface member distally relative to a housing for a dose delivery operation. The user interface member may be moved towards the housing for the dose delivery operation. Of course, as the motion sensing unit is operative only comparatively late, it is particularly advantageous to compare the relative angular positions of the first member and the second member with each other as, then, it can be determined whether there has been relative movement before the motion sensing unit has been operative and, preferably, by how much the members have moved.

In one embodiment, the electronic system is configured such that before the motion sensing unit is rendered operative, the electronic control unit is switched from a first state of low power consumption to a second state of higher power consumption. For example, the electronic control unit may be switched from an off-state where no or reduced power is supplied to the unit to an on-state where more electrical power is supplied. This switching can be triggered by the use signal. After the electronic control unit has been activated, it may issue an activation signal or command to the motion sensing unit such that this unit is switched from the first state into the second state. Alternatively, the use signal may be used to trigger an already active electronic control unit to activate the motion sensing unit or to directly activate the motion sensing unit.

In one embodiment, the comparison operation is configured to determine whether there is an offset between dose information characteristic for the respective operation which is derived from the data signals generated or generatable by the motion sensing unit and the actual dose information characteristic for the dose setting operation or the dose delivery operation. The offset may correspond to or be determined by the difference in the relative angular positions of the first member and the second member between the onset of their relative movement for a particular operation, e.g. for a dose setting operation or a dose delivery operation, and the generation of the first data signal by the motion sensing unit, which may be indicative for the point in time after which the motion sensing unit can quantify the relative movement between the first and second members. Preferably, the comparison operation or its result is configured to or suitable to quantify the offset, e.g. in whole-number multiples of one unit setting increment. Thus, it can be gathered from the comparison operation by how much the first and the second member have rotated relative to each other before the relative rotation could be sensed or monitored by the motion sensing unit. If the comparison of the first data and the second data yields that the first and the second relative angular positions are identical no correction for the dose information needs to be made, i.e. there is no offset or the offset is less than one unit increment. Otherwise, if a difference in the positions is found during comparison, there will be an offset or it will be assumed that there is an offset and appropriate corrections can be made or actions can be triggered. For example, if the result of the comparison operation is that the first relative position and the second relative position are different, the electronic system is configured to consider this difference when calculating dose information characteristic for the dose setting operation or the dose delivery operation. The dose information may be or may relate to the number of units, e.g. the number of international units (IU), which would have been dispensed if a liquid drug of the size of the set dose would have been dispensed during the dose delivery operation. The dose information may be closely related or governed by the number of unit setting increments characterizing the angular distance between an initial relative position of the first member and the second member before the operation is commenced and the end position after the respective operation has been completed.

In one embodiment, the first data may be data indicative for the first relative angular position of the first and second member detectable via the motion sensing unit during a particular operation of a first type, e.g. a dose setting operation or a dose delivery operation. Alternatively or additionally, the second data may be data indicative for an angular end position between the first member and the second member at the end of a previous operation, expediently an operation of the first type, e.g. dose setting operation or dose delivery operation. An intermediate operation of a second type different from the first type may have to occur between the previous operation of which the end position is indicated by the second data and the particular operation. The intermediate operation may be a dose setting operation between two successive dose delivery operations, for example. That is to say, if the first data is data indicative for a position during a dose delivery operation, the second data may be data indicating the end position after a previous dose delivery operation with one, preferably only one, dose setting operation having occurred between the two successive dose delivery operations. The first data may be generated only after the onset of the particular operation. The offset between, e.g. the angular distance between, the first relative angular position and the angular end position may be a difference between stored data (second data) and the first data sensed or sensable by the motion sensing unit or be characterized by that difference. During the intermediate operation, the relative positions between the first member and the second member may be constant, for example. The second data may be stored in the electronic system, preferably at the end of the previous operation, e.g. the dose delivery operation.

In one embodiment, the electronic system is configured such that, due to the comparison operation, an offset of more than one unit setting increment, e.g. two or more than two, or three or more than three unit setting increments, for example up to seven unit setting increments, can be detected and/or quantified. Generally, the limit of the maximum offset which may be detected or compensated by the system may be given by the number of unique sequential relative positions of the first member and the second member which are identifiable by the system minus one.

In one embodiment, the electronic system comprises an electrical use detection unit. The use detection unit may be operatively connected to the electronic control unit, e.g. electrically conductively, such as via a conductor on a conductor carrier. The electrical use detection unit may be configured to generate or trigger a use signal, e.g. an electrical signal or current. The use signal may be indicative that the user has commenced the dose setting operation or the dose delivery operation. Commencement of the dose setting operation or the dose delivery operation may require relative rotational movement between the first member and the second member. When the dose delivery operation has been commenced, a piston rod may be displaced in a dose delivery direction, e.g. distally relative to a housing. Accordingly, the use signal may be generated only after the dose setting operation or the dose delivery operation has been commenced or initiated. In this way, it can be ensured that, when the use signal is generated, an operation it should be indicative for has already been commenced. Alternatively, the use signal may be indicative that the user intends to commence the dose setting operation or the dose delivery operation. Then, it may be generated before the operation is commenced. In this case, the piston rod does not have to move before or when the use signal indicative for the dose delivery operation is generated.

In one embodiment, the electronic system is configured to generate the use signal in response to relative rotational movement between the first member and the second member. In this case, the use signal may be generated after the operation has been commenced already.

In one embodiment, the electronic system is configured such that the motion sensing unit is switched from a first state, in which the motion sensing unit is not operative, into a second state, in which the motion sensing unit is operative by the electronic control unit in response to the use signal. The second state may be an on-state of the motion sensing unit. In the first state, the motion sensing unit may be switched off or have a reduced power consumption compared to the second state. After the respective operation has been completed and/or after a predetermined time has elapsed since the switching to the second state, the electronic control unit may be configured to switch the motion sensing unit back to the first state, in particular if no data signals are generated within the predetermined time.

In one embodiment, in case the result of the comparison operation is that the first and second relative angular positions are identical, it is ensured that the motion sensing unit has been switched to the second state timely enough to sense the relative movement between the first member and the second member from the onset of the dose setting operation or the dose delivery operation.

In one embodiment, the motion sensing unit comprises a sensor arrangement, the sensor arrangement comprising one sensor or a plurality of sensors. The respective sensor may be contactless. The respective sensor may be a radiation detector, for example. However, other sensing technologies could also be applied. The motion sensing unit may comprise an electromagnetic radiation emitter, e.g. an LED. The radiation emitter may emit radiation towards an encoder component and the radiation detector may be arranged and configured to detect radiation reflected from the encoder component. If the region of the encoder component which currently is in a detection position relative to the respective sensor is a detection region, more radiation may be reflected towards the radiation detector and, accordingly, the signal may be higher.

In one embodiment, the electronic system comprises an encoder component. The encoder component may comprise a plurality of, preferably angularly separated, detection regions. The detection regions may be configured to excite the respective sensor of the sensor arrangement, in particular when they are or are being moved into a detection position relative to the respective sensor. Each of the detection regions may be suitable to be moved into detection position relative to the respective sensor, e.g. relative to one sensor, relative to a selected plurality of all sensors or relative to all sensors of the sensor arrangement. Specifically, in a detection region, the sensor signal or data signal generated in the respective sensor of the sensor arrangement may be greater than in non-detection regions between two adjacent detection regions.

In one embodiment, one of the sensor arrangement and the encoder component is associated with the first member, e.g. axially and/or rotationally fixed to the first member, and the other one of the sensor arrangement and the encoder component is associated with the second member, e.g. rotationally and/or axially fixed to the second member.

In one embodiment, the system may be configured to calculate the (current) rotation speed or rotational speed of the relative rotation between the first member and the second member, e.g. based on the time difference between the point in time when the motion sensing unit has become operative and the time when the first data signal is being generated by the motion sensing unit and/or based on the time difference between two subsequent data signals generated during the operation by the motion sensing unit. The time to power-up or render the motion sensing unit operative may be constant, especially after generation of the use signal.

The absolute change in position between the first member and the second member which has occurred before the motion sensing unit became operative may depend on the rotation speed and/or the acceleration. Speed or acceleration, in turn, may depend on the user as the system is preferably user-force drive. The speed and/or the acceleration derivable from one or more data signals of the motion sensing unit may provide a basis for calculating the absolute angular change in position, e.g. in unit setting increments, especially as the power-up time until the motion sensing unit may be active is constant or essentially constant. Thus, based on the calculated rotation speed and the stored second data, the angular distance which the encoder component has rotated before the first data signal has been generated may be calculated, e.g. in the control unit. This angular distance is characteristic for the number of unit setting increments which have been missed or not sensed by the motion sensing unit. Accordingly, the number of missed unit setting increments can be calculated and added to the dose information which is calculated by the electronic system such as by the electronic control unit or other electronic units of the system based on data derived from the motion sensing unit. This calculation of the rotation speed may be applied as an alternative or additionally to the procedure below which uses the number of unique sequential relative position between the first and second members as seen along the rotational direction.

In one embodiment, the sensor arrangement and the encoder component are arranged and configured such that that there is a number N of uniquely identifiable sequential relative angular positions between the encoder component and the sensor arrangement. A potential offset of up to N−1 unit setting increments can be compensated via the comparison operation in case there is a plurality of unique sequential positions which the encoder component can assume relative to the sensor arrangement. The positions may be identifiable via the pattern of the sensor signals (e.g. the data signals), the knowledge in which sensor the particular signal is generated, and/or the configuration of the encoder component.

In one embodiment, the number of sensors of the sensor arrangement, the arrangement of the sensors of the sensor arrangement relative to one another, the arrangement of the detection regions relative to the sensors of the sensor arrangement, and/or the configuration of the detection regions of the encoder component are chosen, e.g. adjusted to one another, such that there is a number N of uniquely identifiable sequential relative angular positions between the encoder component and the sensor arrangement. Within the sequence of the position, e.g. in the rotation direction of the relative rotation, each position may be identifiable via one or more sensor or data signals. Preferably, all of these positions are situated within one full rotation by 360° of the first member relative to the second member or even within less than one full rotation.

In one embodiment, N is greater than or equal to any one of the following values: 2, 3, 4, 5, 6, 7, 8. Alternatively or additionally, N may be less than or equal to any one of the following numbers: 16, 15, 14, 13, 12, 11, 10, 9. N may be between 2 and 16, for example.

In one embodiment, the respective data, i.e. second data and/or first data, may be digital data.

In one embodiment, the sensor arrangement and the encoder component may be adjusted such that the sensor signals (data signals) are suitable to produce or form a Gray code. The Gray code may uniquely identify successive relative angular positions.

In one embodiment, the system is configured such that a sequence of successive relative angular positions between the first member and the second member is uniquely identifiable via a Gray code or Gray code data. Particularly, data on two adjacent positions, preferably on any two adjacent positions, may differ in only one bit.

In one embodiment, the second data comprises only data indicative for one position, e.g. the relative angular end position of the first member and the second member after the dose delivery operation has been completed.

In one embodiment, the second data comprises or is configured to comprise not only data indicative for one position but for a plurality of or a sequence of, preferably sequentially disposed, relative angular positions between the first member and the second member. For example, the second data comprises or is configured to comprise data indicative for a sequence of N relative angular positions. Here, N may be the number of uniquely identifiable angular positions mentioned above. One of these positions in the second data is expediently the end position of the previous dose setting or dose delivery operation. The other positions may be positions preceding the end position in a direction counter to the relative rotation direction. N is preferably greater than or equal to three, e.g. four.

In one embodiment, the first data comprises or is configured to comprise not only data indicative for one position but for a plurality of or a sequence of, preferably sequentially disposed, relative angular positions between the first member and the second member. For example, the first data comprises or is configured to comprise data indicative for a sequence of N relative angular positions. One of these positions in the first data is expediently the first detectable position by the motion sensing unit during the dose setting or dose delivery operation and investigation, e.g. the position derivable from the first data signal or signals. The other positions may be positions which are subsequently assumed by the first member and the second member.

In case a sequence of successive (sequential) positions is available in the first and/or second data, the rotation direction could also be verified easily, for example. Alternatively or additionally, the plurality of positions in the respective data may be used to verify whether the other data is correct and/or the system functions properly.

In one embodiment, the electronic system is configured to determine the angular distance by which the first member and the second member move relative to one another during the dose setting operation or the dose delivery operation based on data signals generated or generatable by the motion sensing unit. The annular distance may be determined in whole-number multiples of the unit setting increment. The angular distance may determine the size of the set or delivered dose, i.e. dose information for the user.

In one embodiment, the electronic system is configured to quantify the relative rotational movement of the first member and the second member in whole-number multiples of one unit setting increment.

In one embodiment, based on the result of the comparison operation and particularly if an offset is detected between the first relative angular position and the second relative angular position, an offset angular distance between the first relative angular position and the second relative angular position, preferably converted into whole-number multiples of the unit setting increment, may be added to the angular distance of the relative movement between the first member and the second member determined via the motion sensing unit, preferably converted into whole-number multiples of the unit setting increment. Based on the total distance the dose information may be calculated.

In one embodiment, the angular extension of the respective detection region is greater than one unit setting increment. The angular extension of the detection regions and of non-detection regions between the detection regions may be equal, particularly when measured in unit setting increments. The angular extension of the respective detection region may be a whole-number multiple of the unit second increment, e.g. an even or odd whole-number multiple, such as two unit setting increments.

In one embodiment, the sensor arrangement comprises two sensors.

In one embodiment, the angular offset between two sensors of the sensor arrangement is an odd whole-number multiple of one unit setting increment or an even whole-number multiple of one unit setting increment.

In one embodiment, the angular offset between two sensors (or the angular offset between the detection positions of or for the sensors) is adjusted with respect to the detection regions such that the sensors (or the angular offset between the detection positions of or for the sensors) are out-of-phase with respect to the detection regions. For example, if the angular extension of the detection region is an even multiple of one unit setting increment, the angular offset between two sensors may be an odd multiple of one unit setting increment. If the angular extension of the detection region is an odd multiple of one unit setting increment, the angular offset between two sensors may be an even multiple of one unit setting increment.

In one embodiment, the electronic system or the drug delivery device comprises a dose setting and drive mechanism. The dose setting and drive mechanism may be configured to perform the dose setting operation and/or the dose delivery operation. The dose setting and drive mechanism may comprise the first member. The electronic system may comprise the second member. The second member may be part of the dose setting and drive mechanism, particularly insofar that it moves either during the dose setting operation or the dose delivery operation or during both operations, or it may be stationary during dose setting and/or during dose delivery, e.g. relative to a housing of the electronic system or the drug delivery device, or, alternatively, it may be the housing. The dose setting and drive mechanism may be configured such that, in the dose delivery operation and/or in the dose setting operation, the first member rotates relative to the second member and/or the housing. The dose setting mechanism may be configured to perform a dose setting operation for setting a dose to be delivered by the drug delivery device. The dose which can be set in the drug delivery device may be a variable dose, i.e. the size of the dose which can be set is not fixed by the design of the mechanism but rather can be chosen by the user. Preferably, the user can choose the set dose between a minimum settable dose and a maximum settable dose. The drive mechanism may be configured to perform a dose delivery operation for delivering the dose, e.g. the dose which has been previously set.

In one embodiment, the dose setting and drive mechanism comprises a user interface member, e.g. dose and/or injection button. The user interface member may be arranged to be operated by the user to operate the mechanism.

In one embodiment, the first member and/or the second member may be configured to move during the dose setting operation and/or the dose delivery operation relative to a housing of the electronic system or the drug delivery device. The first member may be a dose member or dial member of the dose setting and/or drive mechanism which is moved to set a dose, e.g. a dial sleeve or a number sleeve. The second member may be a drive member, e.g. a member engaged with a piston rod of the dose setting and/or drive mechanism, and/or a user interface member, such as a dose and/or injection button. The first member and/or the second member may be movably coupled to or retained in the housing. In the dose setting operation, the first member and/or the second member may be displaced axially relative to the housing, for example away from a proximal end of the housing. The distance by which the first member and/or the second member is displaced during the dose setting operation relative to the housing axially may be determined by the size of the set dose. In other words, the drug delivery device may be of the dial extension type, i.e. the device increases its length during the dose setting operation in an amount proportional to the size of the set dose. However, the present disclosure should not be understood as being restricted to such devices.

In one embodiment, in the dose setting operation and/or in the dose delivery operation, the first member moves, e.g. rotates and/or moves axially, relative to the second member. For example, the first member may rotate relative to the second member during the dose delivery operation. The first member and the second member may both move axially during the dose delivery operation. The second member may be rotationally locked with respect to the housing during the dose delivery operation, e.g. by a delivery clutch. The first member and the second member may be rotationally locked relative to one another during the dose setting operation. Accordingly, the first member and the second member may rotate relative to the housing in the dose setting operation. During the dose setting operation the first member and the second member may be coupled to one another, e.g. via a coupling interface, such as a setting clutch. The coupling interface may rotational lock the first member and the second member to one another during the dose setting operation. When the coupling interface is engaged, the first member and the second member may be rotationally locked with one another, such as by direct engagement of coupling interface features. The first member and the second member may comprise mating coupling interface features. The coupling interface may be released during the dose delivery operation. Particularly the use signal which is explained in more detail below may be generated only when the coupling interface, e.g. the delivery clutch and/or the setting clutch, has changed its state, e.g. from engaged to released or vice versa, and/or after the first member has already rotated relative to the second member.

In one embodiment, the first member and the second member rotate relative to one another during only one of the dose setting operation and the dose delivery operation. One of the first member and the second member may rotate relative to the housing during both operations. One of the first member and the second member may rotate relative to the housing during only one of the operations, e.g. during dose setting or during dose delivery.

In one embodiment, the dose setting and/or drive mechanism comprises a dose member, e.g. a number sleeve or a dial sleeve. The dose member may be rotatable in the dose setting operation in whole-number multiples of a unit setting increment relative to the housing. During the dose setting operation, the dose member may be operatively coupled, e.g. rotationally locked to the user interface member and/or the second member. The unit setting increment may be an angle which is constant. Accordingly, the unit setting increment may define the smallest dose settable with the dose setting and/or drive mechanism. One unit setting increment may correspond to a rotation of greater than or equal to 10° and/or a rotation of less than or equal to 20°, e.g. 15°. The electronic system may comprise an incremented setting interface which defines the unit setting increment. The setting interface may be a ratchet interface. The ratchet interface may be formed between the dose member and the housing, for example.

In one embodiment, the first member is a member different from a user interface member. Specifically, the first member may not have any surface which is intended to be touched by the user for operating the drug delivery device or the system. The first member may be an interior member of the drug delivery device or the system. The first member may be arranged within the housing of the drug delivery device.

In one embodiment, the rotation axis around which one, more, or all of the rotations discussed herein take place may be a longitudinal axis of the housing and/or the rotation axis around which the first member and/or the second member rotate, e.g. relative to the housing, during dose setting and/or dose delivery.

In one embodiment, at least a portion of the second member is received within the first member.

In one embodiment, the first member and/or the second member or a portion of the respective member has a sleeve-like configuration.

In one embodiment, the relative rotation of the first member and the second member during the dose delivery operation may be indicative for the size of the dose being dispensed in the dose delivery operation or being set in the dose setting operation.

In one embodiment, the electronic system is configured such that the rotation between the first member and the second member is unidirectional. Thus, relative rotation between the two members in the other direction may be prevented. This stabilizes the relative position between the first member and the second member. A unidirectional ratchet operatively coupled to or acting between the first member and the second member may be provided for ensuring the unidirectional rotation. Only one of the first and the second members may be rotatable actively relative to the other one during operation of the electronic system or the drug delivery device. Preferably, the first member is rotated. For example, the first member may rotate relative to the second member during only one of the dose setting operation and the dose delivery operation, e.g. during the dose delivery operation. Relative rotation between the first member and the second member may be prevented during the other one of the dose setting operation and the dose delivery operation, e.g. during the dose setting operation.

In one embodiment, the sensor arrangement and the encoder component are arranged and configured such that, when the first member rotates relative to the second member, the encoder component rotates relative to the sensor arrangement.

In one embodiment, the sensor arrangement is rotationally secured to the housing during the dose delivery operation or during the dose setting operation.

In one embodiment, the dose setting and drive mechanism is configured such that after an operation of the respective type, i.e. dose setting operation or dose delivery operation, has been completed, the relative angular position between the first member and the second member is not changed before the subsequent operation of the same type is conducted. For this purpose, a securing system or ratchet system may be provided which defines a stable end position of the two members.

In one embodiment, the drug delivery device comprises a reservoir with a drug and/or a reservoir retainer for retaining a reservoir with a drug in the drug delivery device.

Further aspects, embodiments and advantages will become apparent from the following description of the exemplary embodiments in conjunction with the drawings.

### BRIEF DESCRIPTION OF THE FIGURES

FIG. 1 shows an embodiment of a drug delivery device.

FIG. 2 shows a proximal end of a drug delivery device according to another embodiment.

FIG. 3A shows a proximal end of the injection device of FIG. 2 after actuation of an injection button.

FIG. 3B is a cross-sectional view of the device of FIG. 2 after actuation of the injection button.

FIG. 4 is a zoomed-in cross-sectional view of the device of FIG. 2.

FIG. 5 is an elevated side view of a first type of encoder system.

FIG. 6 is a plan view of the encoder system shown in FIG. 5.

FIG. 7 is a schematic block diagram of a device controller.

FIG. 8A is a cross-sectional view of the proximal end of a device before actuation of an injection button.

FIG. 8B is a cross-sectional view of the proximal end of a device during partial actuation of an injection button.

FIG. 8C is a cross-sectional view of the proximal end of a device during full actuation of an injection button.

FIG. 9 is an elevated side view of a second type of encoder system.

FIG. 10 is a plan view of the encoder system shown in FIG. 9.

FIG. 11 illustrates a gray code output.

FIG. 12 is a partial plan view of an encoder system.

FIG. 13 is a partial plan view of an encoder system.

FIG. 14 is an elevated side view of a third type of encoder system.

FIG. 15A is a partial plan view of an encoder system.

FIG. 15B is a partial plan view of an encoder system.

FIG. 16 is an elevated side view of a fourth type of encoder system.

FIG. 17 is an elevated side view of a fifth type of encoder system.

FIG. 18A is a plan view of a sixth type of encoder system.

FIG. 18B is a plan view of a seventh type of encoder system.

FIG. 19A is a screenshot showing scope traces obtained from various embodiments.

FIG. 19B is a close-up view of the screenshot of FIG. 19A.

FIG. 20 illustrates schematically an embodiment of an electronic system for a drug delivery device.

FIG. 21 illustrates an embodiment of the electronic system.

FIG. 22 illustrates another embodiment of the electronic system.

FIG. 23 illustrates another embodiment of the electronic system.

FIG. 24 illustrates another embodiment of the electronic system.

FIG. 25A to 25C illustrates another embodiment of the electronic system.

FIG. 26 illustrates another embodiment of the electronic system.

### DETAILED DESCRIPTION

In the figures, identical elements, identically acting elements or elements of the same kind may be provided with the same reference numerals.

In the following, some embodiments will be described with reference to an insulin injection device. The present disclosure is however not limited to such application and may equally well be deployed with injection devices that are configured to eject other medicaments or drug delivery devices in general, preferably pen-type devices and/or injection devices.

Embodiments are provided in relation to injection devices, in particular to variable dose injection devices, which record and/or track data on doses delivered thereby. These data may include the size of the selected dose and/or the size of the actually delivered dose, the time and date of administration, the duration of the administration and the like. Features described herein include the arrangement of sensing elements and power management techniques (e.g. to facilitate small batteries and/or to enable efficient power usage).

Certain embodiments in this document are illustrated with respect to Sanofi's AllSTAR® injection device where an injection button and grip (dose setting member or dose setter) are combined. The injection button may provide the user interface member for initiating and/or performing a dose delivery operation of the drug delivery device. The grip or knob may provide the user interface member for initiating and/or performing a dose setting operation. Both devices are of the dial extension type, i.e. their length increases during dose setting. Other injection devices with the same kinematical behaviour of the dial extension and button during dose setting and dose expelling operational mode are known as, for example, the Kwikpen® or Savvio® device marketed by Eli Lilly and the Novopen®4 device marketed by Novo Nordisk. An application of the general principles to these devices therefore appears straightforward and further explanations will be omitted. However, the general principles of the present disclosure are not limited to that kinematical behaviour. Certain other embodiments may be conceived for application to Sanofi's SoloSTAR® injection device where there are separate injection button and grip components/dose setting members. Thus, there may be two separate user interface members, one for the dose setting operation and one for the dose delivery operation.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

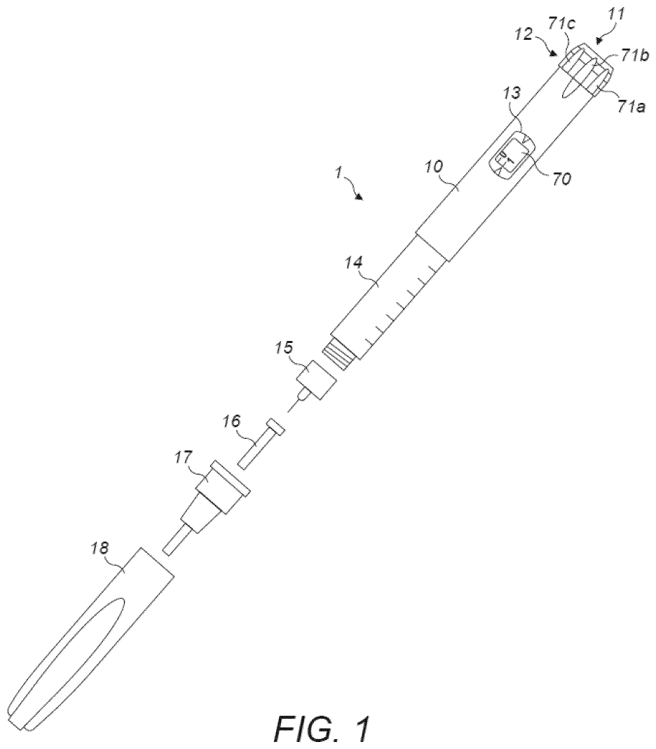

FIG. 1 is an exploded view of a medicament delivery device or drug delivery device. In this example, the medicament delivery device is an injection device 1, e.g. a pen-type injector.

The injection device 1 of FIG. 1 is an injection pen that comprises a housing 10 and contains a container 14, e.g. an insulin container, or a receptacle for such a container. The container may contain a drug. A needle 15 can be affixed to the container or the receptacle. The container may be a cartridge and the receptacle may be a cartridge holder. The needle is protected by an inner needle cap 16 and either an outer needle cap 17 or another cap 18. An insulin dose to be ejected from injection device 1 can be set, programmed, or ‘dialled in’ by turning a dosage knob 12, and a currently programmed or set dose is then displayed via dosage window 13, for instance in multiples of units. The indicia displayed in the window may be provided on a number sleeve or dial sleeve. For example, where the injection device 1 is configured to administer human insulin, the dosage may be displayed in so-called International Units (IU), wherein, for insulin, one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming.

In this example, the dosage knob 12 includes one or more formations 71a, 71b, 71c to facilitate attachment of a data collection device.

The injection device 1 may be configured so that turning the dosage knob 12 causes a mechanical click sound to provide acoustical feedback to a user. In this embodiment, the dosage knob or dose button 12 also acts as an injection button 11. When needle 15 is stuck into a skin portion of a patient, and then dosage knob 12/injection button 11 is pushed in an axial direction, the insulin dose displayed in display window 13 will be ejected from injection device 1. When the needle 15 of injection device 1 remains for a certain time in the skin portion after the dosage knob 12 is pushed home, the dose is injected into the patient's body. Ejection of the insulin dose may also cause a mechanical click sound, which is however different from the sounds produced when rotating the dosage knob 12 during dialing of the dose.

In this embodiment, during delivery of the insulin dose, the dosage knob 12 is returned to its initial position in an axial movement, without rotation, while the dial sleeve 70 is rotated to return to its initial position, e.g. to display a dose of zero units. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g. 28 days after the first use) is reached. If the injection device is a reusable device, then the insulin container can be replaced.

Furthermore, before using injection device 1 for the first time, it may be necessary to perform a so-called “prime shot” to ensure fluid is flowing correctly from insulin container 14 and needle 15, for instance by selecting two units of insulin and pressing dosage knob 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

As explained above, the dosage knob 12 also functions as an injection button 11 so that the same component is used for dialling/setting the dose and dispensing/delivering the dose.

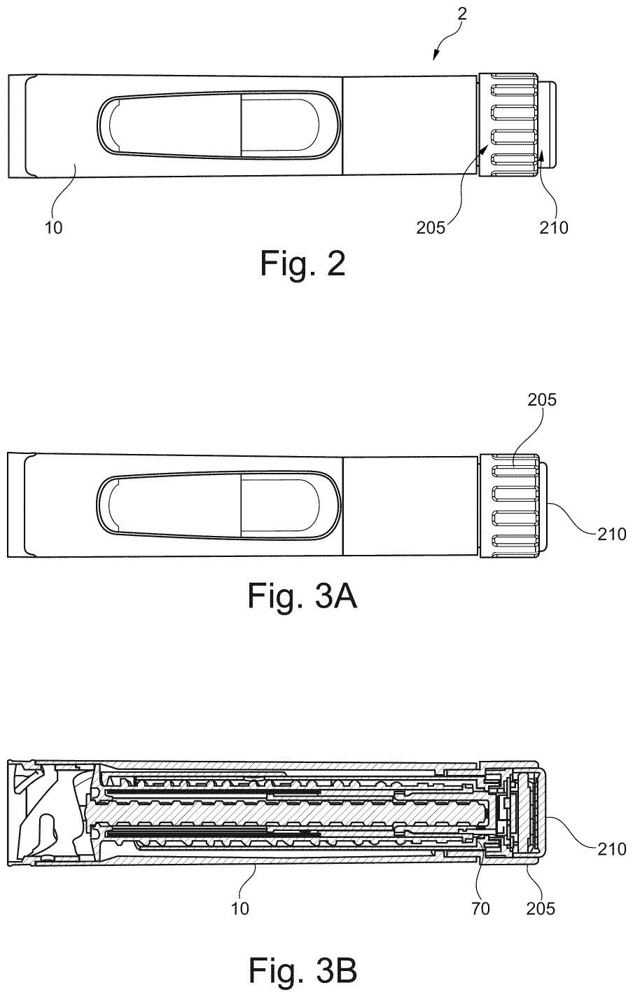

FIGS. 2, 3A and 3B show the proximal end of a device 2 according to a second embodiment. The device 2 comprises a grip 205 and injection button 210. Unlike the device 1 shown in FIG. 1, the injection button 210 is separate from the grip 205 which is used to dial the dosage. The dial sleeve 70 and injection button 210 are located partially inside the grip 205. The grip 205 and dial sleeve 70 may be considered functionally as elements of the same component. Indeed, the grip 205 and dial sleeve 70 may only be separate components for assembly reasons. Aside from the differences described herein, the device 2 shown in FIG. 2 operates in substantially the same way as the device 1 shown in FIG. 1.

Similarly to the device 1, the dial sleeve 70, grip 205 and injection button 210 extend helically from the device 2. During a dose-dialling mode of operation (as shown in FIG. 2) there is no relative rotation between the injection button 210 and the dial sleeve 70. The dose is dialled by rotating the grip 205 (thereby also rotating the dial sleeve 70 and injection button 210) with respect to the rest of the device 2.

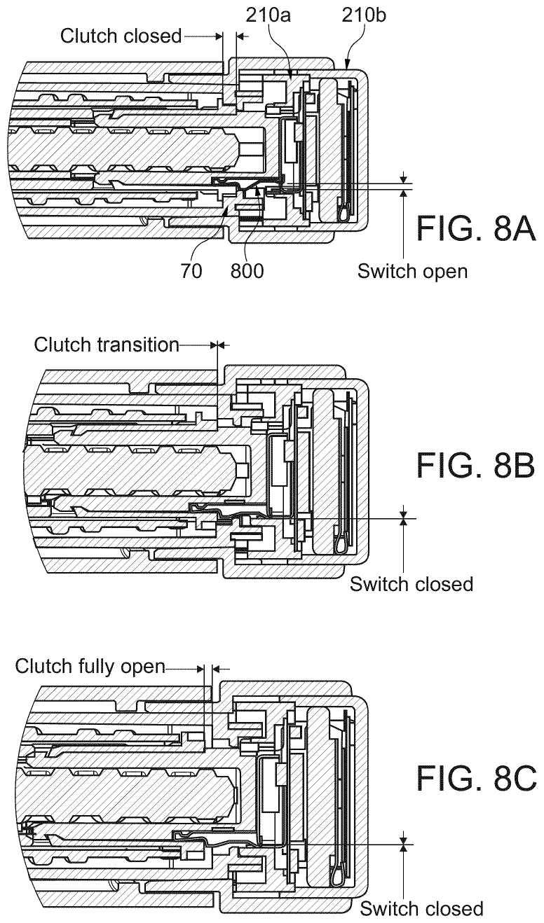

To initiate dispensing of a medicament, the injection button 210 is pressed axially, as shown in FIGS. 3A and 3B. This action changes the mode of the device 2 to a dispensing mode. In dispensing mode the dial sleeve 70 and grip component 205 retract along a helical path into the rest of the device 2, whereas the injection button 210 does not rotate and only retracts with axial motion. Thereby, in dispensing mode, there is a disengagement of the injection button 210 leading to relative rotation of the injection button 210 with respect to the dial sleeve 70. This disengagement of the injection button 210 with respect to the dial sleeve 70 is caused by a clutch arrangement or interface described in more detail in relation to FIGS. 8A-C.

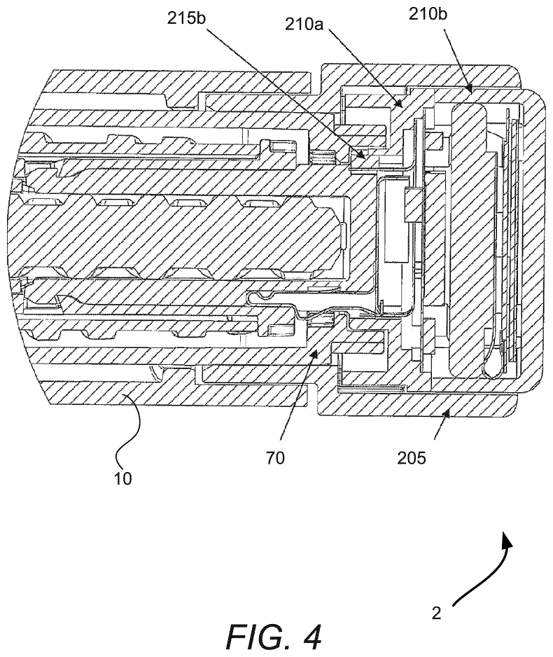

FIG. 4 is a close-up cross sectional view of the proximal end of the device 2 shown in FIG. 3 after the injection button 210 has been pressed. As shown in FIG. 4, the injection button 210 is configured as two separate sub-components, namely a distal or lower button part 210a and a proximal or upper button part 210b. The injection button 210 may be configured in this way to aid the assembly process. The distal button part 210a and proximal button part 210b may be fixed together and act functionally as a single component, i.e. the injection button 210.

A sensor arrangement 215 comprising one or more sensors is mounted in the injection button 210 which is configured to sense the relative rotational position of the dial sleeve 70 relative to the injection button 210. This relative rotation can be equated to the size of the dose dispensed and used for the purpose of generating and storing or displaying dose history information. The sensor arrangement 215 may comprise a primary sensor 215a and a secondary sensor 215b. In FIG. 4, only the secondary sensor 215b is shown. In the following discussion, these sensors are optical sensors however multiple alternative options are equally applicable to various embodiments such as photoelectric sensors, inductive sensors, capacitive sensors, contact sensors, contactless sensors, magnetic sensors and so forth.

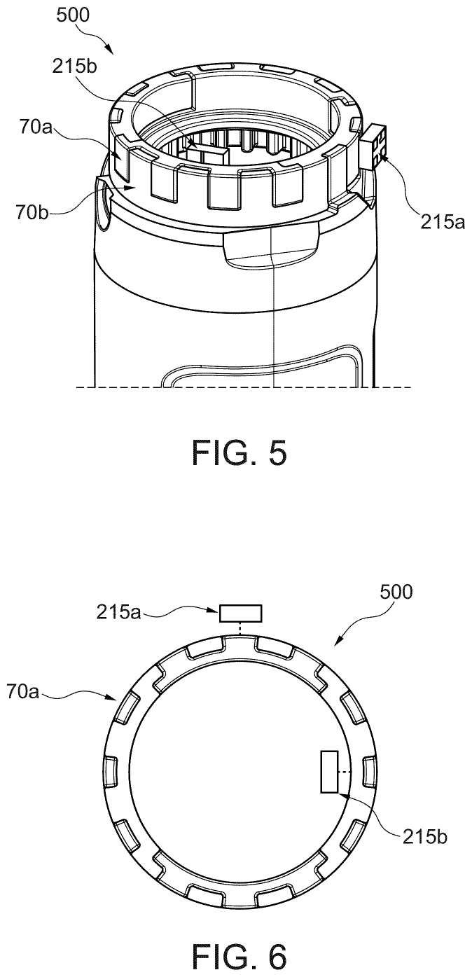

FIGS. 5 and 6 show an encoder system 500 according to certain embodiments. The encoder system is configured for use with the device 2 described above. As shown in FIG. 5 and FIG. 6, the primary sensor 215a and secondary sensor 215b are configured to target specially adapted regions at the proximal end of the dial sleeve 70. In this embodiment, the primary sensor 215a and secondary sensor 215b are infrared (IR) reflective sensors. Therefore, the specially adapted proximal regions of the dial sleeve 70 are divided into a reflective area 70a and a non-reflective (or absorbent) area 70b. The part of the dial sleeve 70 comprising the reflective area 70a and a non-reflective (or absorbent) area 70b may be termed an encoder ring.

To keep production costs to a minimum, it may be favourable to form these areas 70a, 70b from injection moulded polymer. In the case of polymer materials, the absorbency and reflectivity could be controlled with additives, for example carbon black for absorbency and titanium oxide for reflectivity. Alternative implementations are possible whereby the absorbent regions are moulded polymer material and the reflective regions are made from metal (either an additional metal component, or selective metallisation of segments of the polymer dial sleeve 70).

Having two sensors facilitates a power management technique described below. The primary sensor 215a is arranged to target a series of alternating reflective regions 70a and non-reflective regions 70b at a frequency commensurate with the resolution required for the dose history requirements applicable to a particular drug or dosing regime, for example, 1 IU. The secondary sensor 215b is arranged to target a series of alternating reflective regions 70a and non-reflective regions 70b at a reduced frequency compared to the primary sensor 215a. It should be understood that the encoder system 500 could function with only a primary sensor 215a to measure the dispensed dose. The secondary sensor 215b facilitates the power management technique described below.

The two sets of encoded regions 70a, 70b are shown in FIGS. 5 and 6 concentrically with one external and the other internal. However, any suitable arrangement of the two encoded regions 70a, 70b is possible. Whilst the regions 70a, 70b are shown as castellated regions, it should be borne in mind that other shapes and configurations are possible.

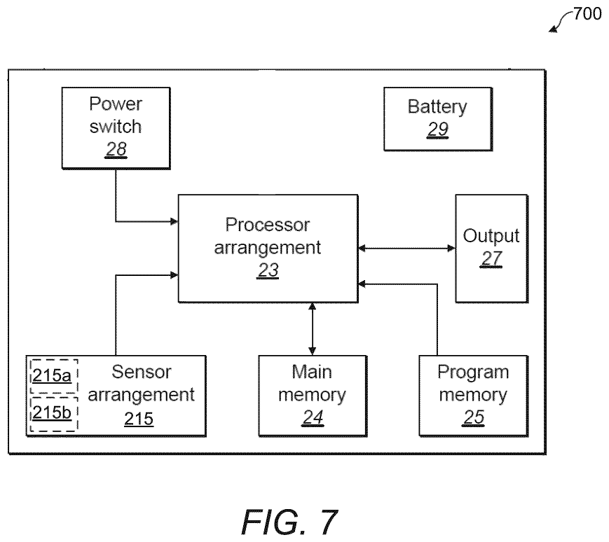

The devices 1, 2 also include a controller 700, as shown schematically in FIG. 7. The controller 700 comprises a processor arrangement 23 including one or more processors, such as a microprocessor, a Digital Signal Processor (DSP), Application Specific Integrated Circuit (ASIC), Field Programmable Gate Array (FPGA) or the like, together with memory units 24, 25, including program memory 25 and main memory 24, which can store software for execution by the processor arrangement 23.

The controller 700 controls a sensor arrangement 215, comprising one or more sensors 215a, 215b.

An output 27 is provided, which may be a wireless communications interface for communicating with another device via a wireless network such as Wi-Fi or Bluetooth®, or an interface for a wired communications link, such as a socket for receiving a Universal Series Bus (USB), mini-USB or micro-USB connector. For example, data may be output to a data collection device attached to the device 1, 2.

A power switch 28 is also provided, together with a battery 29 as power supply.

It is advantageous to be able to minimise the power usage of the encoder system 500 so that the size of a battery 29 needed to be packaged into the device 1, 2 can be minimised. The sensors 215a, 215b used in this embodiment require a certain amount of power to operate. This embodiment is arranged such that the sensors 215a, 215b can be switched on and off intermittently at a controlled frequency (i.e. in a strobe-sampling mode). There is inherently a limit to the maximum rotational speed that can be counted by a sampled encoder system before aliasing occurs. Aliasing is the phenomenon where the sampling rate is less than the rate at which sensed regions pass the sensor which means that a miscount could occur when a region change is missed. The secondary sensor 215b with a reduced frequency compared to the primary frequency 215a can tolerate a higher rotational speed before it too becomes aliased. Whilst the secondary sensor 215b is not able to resolve the dose dispensed to the same resolution as the primary sensor 215a, the output of the secondary sensor 215b remains reliable at higher speeds. Therefore both sensors 215a, 215b are used in combination to be able to accurately determine dose delivered up to a first threshold rotational (dispensing) speed. The sensors 215a, 215b can then be used to determine an approximate dose delivered up to a second (higher) threshold dosing speed. At speeds above the second threshold speed the sensors 215a, 215b will not be able to accurately or approximately determine the dose delivered, therefore the second threshold is set above a speed which is not physically possible when ejecting fluid from the injection device 1, 2.

The first speed threshold is determined by the sampling rate of primary sensor 215a and the frequency of encoder region transitions, which is fixed at the resolution required by the intended drug or dosing regime (for example one transition per 1 IU). The second speed threshold is determined by the sampling rate of the secondary sensor 215b and the frequency of encoder region transitions. The first threshold is set such that the largest range of dispensing speeds can be covered by the system for accurate reporting of dose dispensed.

The example embodiment shown in FIG. 6 has primary sensor 215a targeting region transitions at 1 transition per 1 IU of dose delivered and the secondary sensor 215b targeting region transitions at 1 transition per 6 IU of dose delivered. Other options are possible which include 1 transition per 2 IU, 1 transition per 4 IU, 1 transition per 8 IU and 1 transition per 12 IU units. These options are each possible because there are 24 separate regions 70a, 70b per revolution in the encoder system 500 shown in FIG. 6. In general, if the number of separate regions 70a, 70b per revolution were n units then there would be options at one region transition per m units where m was any integer factor of n greater than 1 and less than n.

The slower the sampling frequency of both sensors 215a, 215b, the lower the power consumption required and therefore the smaller the required size of the battery 29. It is therefore optimal to minimise, by design, the sampling frequency as far as is practical.

In order to further limit the battery capacity requirement, it is advantageous to be able to have the device 2 in a low power state when the sensors 215a, 215b are not required to be energised. This may be achieved with a switch activated by the displacement of the injection button 210.

As shown in FIG. 8A, a switch 800 is mounted in the injection button 210. In the configuration shown in FIG. 8A, an arm of the switch 800 is deflected by the dial sleeve 70 so that the switch 800 is in an open state. In this configuration a clutch between a clutch component and the dial sleeve 70 is engaged with the device 2 in its dialling mode. As the injection button 210 is pressed the injection button 210 is displaced axially with respect to the dial sleeve 70, therefore the switch 800 is displaced axially relative to the dial sleeve 70. This displacement causes a part on the dial sleeve 70 to ride down a cam surface on the switch 800, allowing the switch arm to deflect towards its free state. This deflection in the switch arm has the effect of changing the electrical state of the switch 800 (for example to electrically closed). The design is arranged such that the electrical change of state of the switch 800 happens before the state change in the clutch between the clutch component and dial sleeve 70. FIG. 8B shows the transition point of the clutch and shows that the switch 800 has already changed state. FIG. 8C shows the state of the device 2 with the injection button 210 fully pressed. In this condition, the clutch is fully separated allowing the clutch component and dial sleeve 70 to rotate relative to each other in the dispense mode.

This sequence operates in reverse when the injection button 210 is released.

The change in electrical state that occurs when the injection button 210 is pressed thereby allowing the device 2 to be powered down into a low energy consumption state when the injection button 210 is not pressed. Relative rotation between the injection button 210 and dial sleeve 70 is not possible in this state when the injection button is not pressed, therefore the encoder system 500 is not required.

It is possible for the mechanical configuration between the dial sleeve 70 and the switch 800 to operate in the opposite sense such that the arm of the switch 800 is deflected during dispensing rather than during dialling.

The following embodiments relate to an alternative sensing technique to determine the number of medicament units that have been dispensed from the device 1, 2.

As with the embodiments described above, two sensors 215 are mounted in the injection button 210 and are configured to sense the relative rotational position of the dial sleeve 70 relative to the injection button 210 during the dispensing of a dose. This relative rotation can be equated to the size of the dose dispensed and used for the purpose of generating and storing or displaying dose history information.

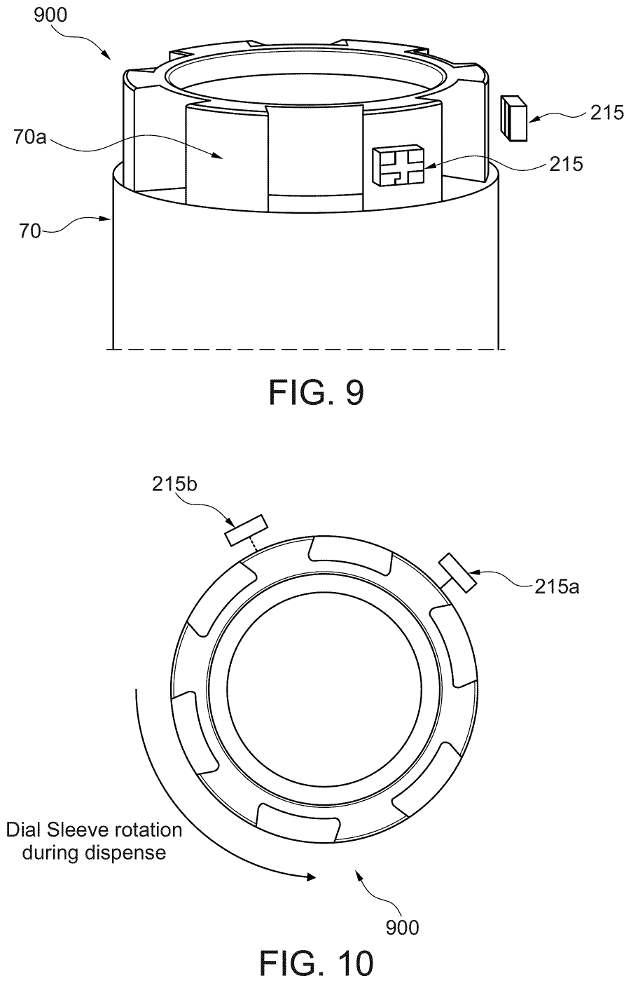

As shown in FIG. 9, the two sensors 215 from this embodiment are configured to target specially adapted regions 70a, 70b of the dial sleeve 70. In this embodiment IR reflective sensors are used, therefore the regions of the dial sleeve 70 are divided into reflective and absorbent segments 70a, 70b. The segments 70a, 70b may also be referred to herein as flags or detection regions.

Unlike the encoder system 500 described above in relation to FIGS. 5 and 6, the encoder system 900 shown in FIGS. 9 and 10 has both IR sensors 215 target the same type of region 70a, 70b. In other words, the sensors 215 are arranged so that they face reflective regions 70a and absorbent regions 70b on the same surface. The sensors 215 may be arranged such that, at the same time, one sensor faces a reflective region 70a and one sensor faces an absorbent region. During the dispensing of a dose, the dial sleeve 70 rotates anti-clockwise 15° relative to the injection button 210 for every medicament unit that has been dispensed. The alternate flag elements are in 30° (or two unit) sections. The sensors 215 are arranged to be out of phase with each other, such that the angle between them equates to an odd number of units (e.g. 15°, 45°, 75°, etc.), as shown in FIG. 10.

The encoder system 900 shown in FIG. 10 has 12 segments or sections per revolution, i.e. 12 alternating regions 70a, 70b. In general, embodiments work with any multiple of 4 units per revolution. The angle, a, between sensors 215 can be expressed by Equation 1, where both m and n are any integers and there are 4 m units dispensed per revolution.

$$α = ( 2 n - 1 ) \frac{360}{4 m} \qquad Equation 1 - Angle between sensors$$

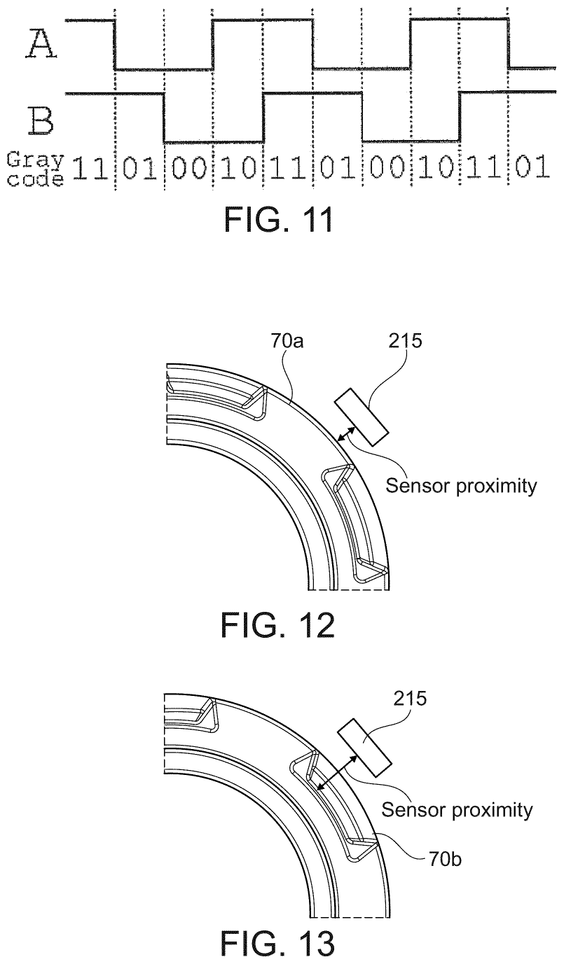

FIG. 11 shows how the outputs for a Sensor A and Sensor B change as the dial sleeve 70 rotates anti-clockwise during dispensing of a medicament.

In combination, the two sensors A, B produce a 2-bit Gray code output (11, 01, 00, 10). The 2-bit code sequence repeats every four units dispensed. This coded output facilitates the detection of positive (anticlockwise) and negative (clockwise) rotations. For example, when the sensors read ‘11’ a change to ‘01’ would be a positive rotation and the change to ‘10’ would be a negative rotation. This directionally sensitive system has advantages over a purely incremental system, in the ability to accurately determine true dispensed dose volume in the cases where negative rotations can occur. For example, in mechanisms that over rotate at the end of dose stop before ‘backing-off’ when the user releases the injection button 210.

Referring to FIG. 12, the IR sensors 215 emit IR light from an LED. The IR reflective regions 70a of the encoder system 900 reflect the light and the sensors detect the reflected light. The sensors 215 then convert the detected light to an electrical output. The strength of the IR light that is detected by the sensor 215 after reflecting off the encoder ring is proportional to the proximity of the sensor to the encoder ring. Therefore it is desirable for the sensor 215 to be as radially close to the encoder ring as possible without contacting the encoder ring, which would add frictional losses to the dispense mechanism.

Referring to FIG. 13, the IR absorbent regions 70b of the dial sleeve 70 do not completely absorb all the IR light emitted from the sensor 215. Testing shows that when the sensor 215 is aligned with the absorbent regions 70b of the dial sleeve 70 the sensors 215 have some electrical output due to the low level of IR light reflected by the dial sleeve 70. Therefore, the dial sleeve flags have been designed to maximise the distance between the sensor 215 and any intentionally IR absorbent parts of the encoder ring. This ensures a high contrast ratio and signal sharpness.

As a dose is dispensed, the software of the device 1, 2 monitors the electrical output of the sensors 215. The software detects changes between high and low outputs to determine when the relative rotation between the dial sleeve 70 and injection button 210 has reached an additional 15° (i.e. an additional one unit has been dispensed). Therefore it is beneficial to the function of the device for the contrast ratio between the high and low outputs to be as large as possible.

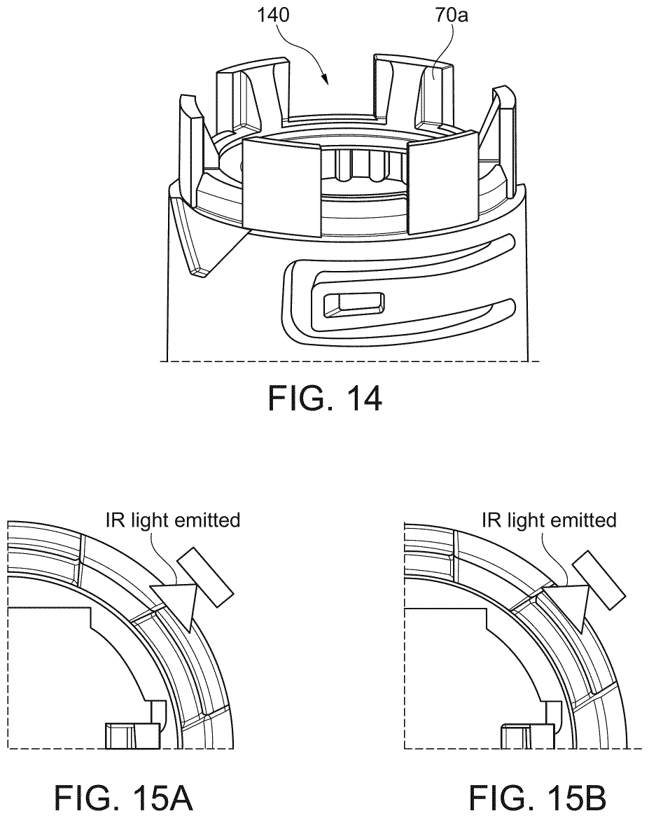

According to various embodiments, the design of the dial sleeve 70 and encoder ring flags 70a, 70b has been developed to increase the contrast ratio. The design shown in FIG. 14 has the absorbent dial sleeve flags 70b removed to leave gaps 140 between adjacent encoder ring flags 70a. This maximises the distance between the sensor 215 and any material which could reflect any of the IR light emitted from the sensor.

This design increases the contrast ratio between the low and high sensor electrical outputs. However, as FIG. 15A shows, the IR light emitted by the sensor 215 is not a beam, such that as the dial sleeve 70 rotates between a reflective encoder ring flag 70a and a gap 140, there is overlap where the sensor 215 detects some of the light emitted by the sensor 215. During this period, the sensor output gradually decreases from high to low, rather than an immediate step change between high and low. This gradual decrease is more difficult for the software to determine as a 15° rotation (i.e. one medicament unit dispensed), than an immediate step change.

This phenomenon occurs with various embodiments of the encoder flags (as shown in FIG. 9 and FIG. 14). However, as shown in FIG. 15B, in accordance with certain embodiments, the rotation of the dial sleeve 70 that is required before the sensor output completely switches to a low output is increased due to the visibility of the sides of the reflective encoder ring flags 70a.

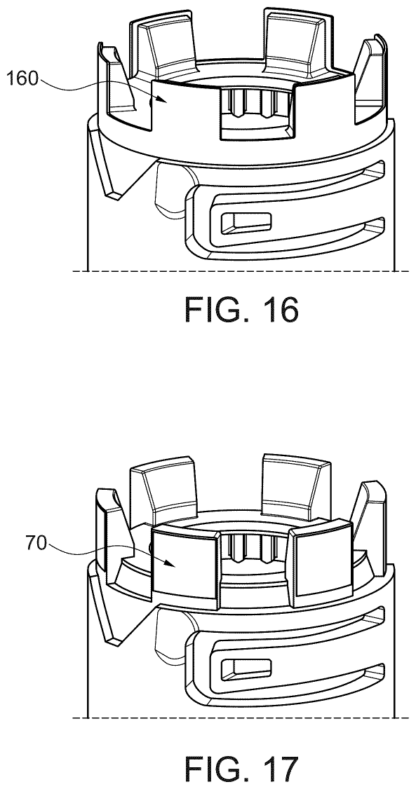

Therefore, it is advantageous to reduce the thickness at the edges of the IR reflective flags 70a on the encoder ring. FIG. 16 and FIG. 17 show two possible embodiments to reduce the thickness at the lateral edges of the IR reflective flags 70a on the encoder ring so that the reflective surfaces are inclined inwardly for preventing or reducing scattered reflection, thereby enhancing contrast transition and signal sharpness.

FIG. 16 shows an embodiment where the moulded polymer encoder ring has been replaced with a formed metal ring 160.

FIG. 17 shows an embodiment where the moulded polymer encoder ring has been replaced by sections of the dial sleeve 70 that have been printed, painted or coated with IR reflective material.

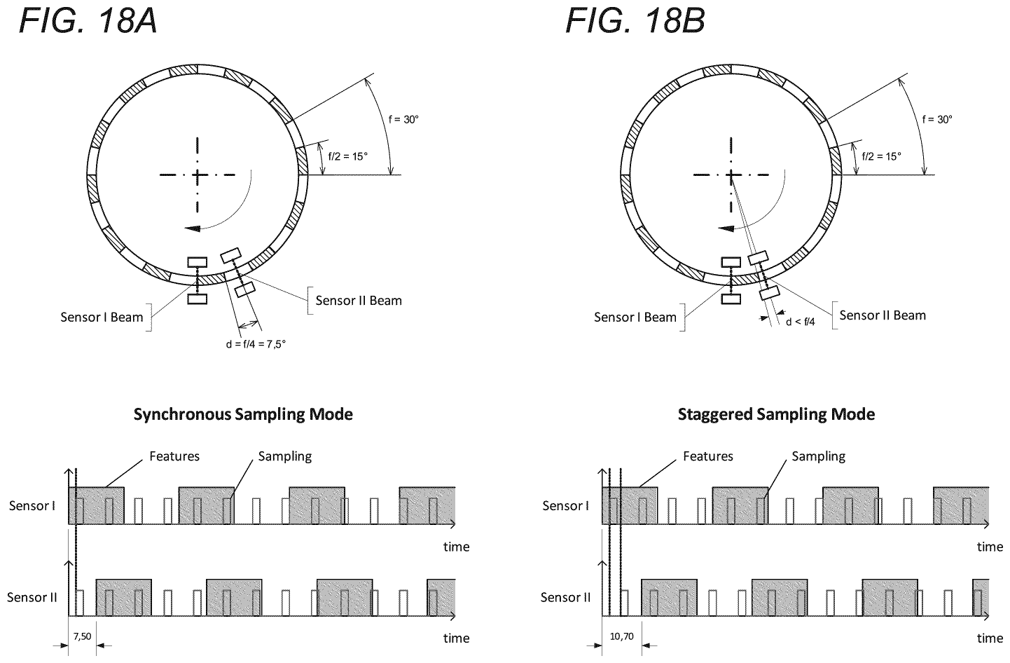

FIGS. 18A and 18B illustrate two alternative modes of operation in accordance with various embodiments. Referring to FIG. 18A, Sensor I and Sensor II are provided having an angular offset (δ) is half of the periodicity (ϕ)) of the encoded regions of the encoder ring. In this embodiment, the sensors are operated to sample synchronously, i.e. at the same times (t₁, t₂, t₃, . . . ).

FIG. 18A illustrates an embodiment where the angular offset (δ) differs from half of the feature periodicity (ϕ/2) and the sensors are operated in a staggered mode with an offset in time (Δt) between samplings. This may be used to achieve more balanced overall system LED power consumption than available in synchronous operation.

In the configuration shown in FIG. 18B, the amount of the angular offset (δ) may be decreased below half of the feature periodicity (ϕ) in order to compensate for the relative angular travel during the offset in time (Δt) between the sampling operations of the different sensors.

The offset in time (Δt) may be adjusted according to an estimated value for the relative rotational speed (ω) of the encoder ring which may be calculated from the sensor measurements. In particular, the offset time (Δt) may be decreased when an increase in rotational speed (ω) is determined.

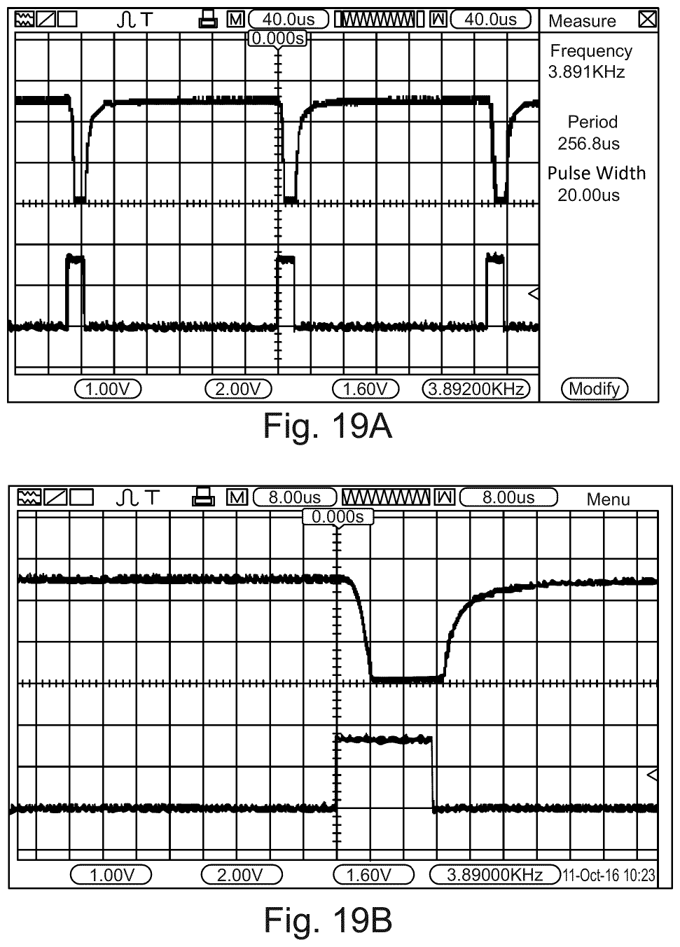

FIG. 19A shows scope traces obtained by embodiments of the disclosure. The lower trace is the LED driving signal and the upper trace is the output from the current mirror before the Schmitt trigger.

FIG. 19B is a zoomed-in view of the scope traces shown in FIG. 19A. Results show that it is possible to sample at 256 μs with a nearly 12-to-1 duty cycle (meaning the average current is 1/12^{th} of the 4 mA LED drive, thereby saving power and cell capacity. This is equivalent to a sample rate of over 3900 Hz and with one unit per segment and a minimum of two samples per segment a detection speed in excess of 1950 units per second is achieved without violating the Nyquist criterion. As such, no anti-aliasing detector is required.

While the embodiments above have been described in relation to collecting data from an insulin injector pen, it is noted that embodiments may be used for other purposes, such as monitoring of injections of other medicaments, for example, or drug delivery devices in general.

As has been discussed above, managing electrical power consumption or the resources of a power supply (e.g. a rechargeable or non-rechargeable battery 29) in drug delivery devices comprising electronic systems, such as the injection devices discussed further above, is a problem which needs to be addressed, e.g. in order to optimize the use of the capacity of the power supply and/or in consideration of the sometimes considerable shelf time of a drug delivery device before the device reaches the user or patient. It needs to be ensured that the device including the electronic system still functions properly for the duration of its intended use.

The present disclosures presents various concepts which can be implemented in drug delivery devices or electronic systems thereof or therefore, e.g. for improving the power management in the devices. Some concepts rely on providing electrical power to certain units of the device only when needed or when it is very likely that the power will be needed. The device which has been discussed above, for example, energizes a motion sensing unit (the sensor system) of the device only when the injection button (as user interface member) is being pressed for performing a dose delivery (injection) operation. The encoder component or encoder ring, after the motion sensing unit has been energized, can be used to gather data on movements which are indicative for the dose which has been delivered during the delivery operation. From the measured movement data, it can be calculated, how much of the drug has actually been delivered. The amount of the actually delivered drug does not necessarily coincide with the dose which was previously set in a dose setting operation, e.g. when the user interrupts the delivery operation before it has been actually completed. Accordingly it is advantageous to measure movements occurring during the dose delivery operation which are correlated to the amount of drug which has been delivered already, e.g. to get insight on the current status or the progress of the delivery operation. The determined delivered dose may be communicated, preferably wirelessly to an external or remote device, e.g. a hand-held device such as a smartphone. In this way a dose log on the doses delivered by the user may be established, which may be accessed by the user easily.

The proposed concepts are suitable for a large variety of drug delivery devices comprising electronic systems or for electronic systems for such devices not only for the devices described further above. The device may be an injection device and/or a pen-type device. The device may be configured to receive or comprise a medicament container or cartridge. The container or cartridge may be filled with liquid drug to be delivered by the device. The device may be designed to deliver a plurality of doses of the drug. Consequently, the container or cartridge may comprise drug in amount sufficient for several doses to be delivered by the device. The device may be re-usable or disposable, where a re-usable device may be provided with a replacement medicament container or cartridge when the current container or cartridge is considered empty or needs to be replaced for different reasons. A disposable device may be a single use device which is disposed after the medicament container has been emptied. The device may be a device of a dial extension type, that is to say a device which increases in length during the dose setting operation, where the increase in length is proportional to the size of the set dose. During the associated dose delivery operation, the length of the device may be decreased again, e.g. until the device resumes its original length, i.e. the length it had before the dose setting operation has been commenced. Alternatively, the length of the device may be independent of the size of the set dose, e.g. constant or substantially constant during dose setting and/or dose delivery. The dose setting operation may involve a, preferably rotational, movement, of a dose setting member as user interface member, e.g. a knob, button or grip component (as discussed further above already). The dose delivery operation may involve a, preferably axial, movement of a dose delivery member as user interface member, e.g. a button such as the injection button discussed further above. As already discussed further above, the dose setting member and the dose delivery member may be formed by a single, e.g. unitary, component, where, preferably, different surfaces of the component are manipulated during the dose setting operation and the dose delivery operation or, alternatively, the dose setting member and the dose delivery member may be separate components/interface members or parts with relative movement being possible between these members, e.g. to switch the dose setting and drive mechanism between a dose setting configuration and a dose delivery configuration. There may be relative movement between these components either during dose setting or dose delivery or during both operations. During the dose setting operation a lateral or side surface, i.e. a radially facing surface, of the dose setting member may be gripped by the user, e.g. with the thumb and index finger. During the dose delivery operation an axially, e.g. proximally, facing surface of the dose delivery member may be touched by the user, e.g. with the thumb. During the dose delivery operation, an axial force may be transferred by the user to the dose delivery member in order to initiate and/or to continuously drive the dose delivery operation using a dose setting and drive mechanism of the device which aside, from the user interface member may comprise further members, such as a drive member and a piston rod, for example. The drive member may engage the piston rod. In one embodiment, the dose delivery member may be the drive member which engages the piston rod, e.g. threadedly. The device may be a device as disclosed, for example, in WO 2015/028439 A1 which is incorporated herein by reference in its entirety.

The device may be a needle-based device, i.e. the drug may be delivered into the body via a needle piercing the skin or may be needle-free. The device may be a device with a delivery assist, e.g. a spring-assisted or spring-driven device. In such devices, the dose delivery operation by the user is assisted or entirely driven by energy provided by an energy storage member such as a spring. The energy in the storage member may be increased during the dose setting operation by the user or the energy storage member may be provided with the entire energy required to empty the medicament container pre-stored in the member by the manufacturer. In the latter case, the user does not need to provide energy to increase the energy stored in the energy storage member such as during the dose setting operation.

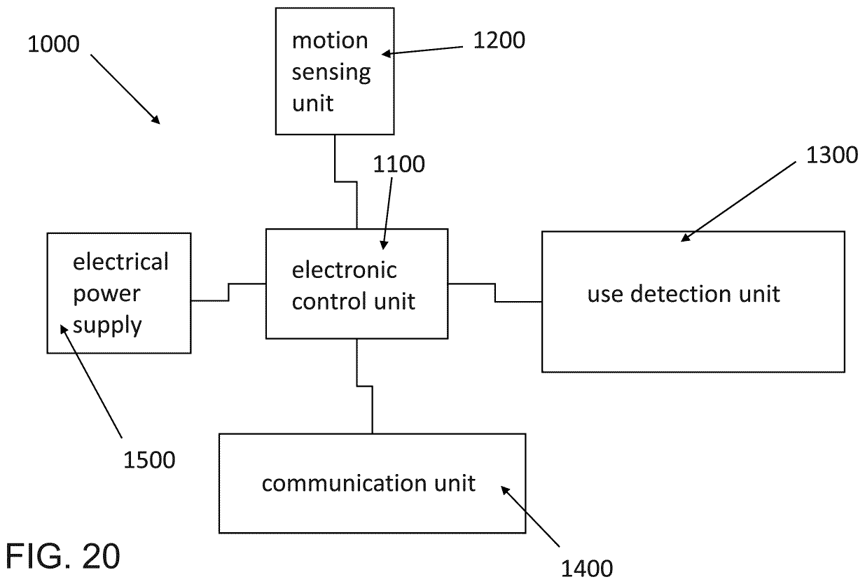

FIG. 20 illustrates a general configuration of elements of an electronic system 1000 which can be used in a drug delivery device, for example one of the devices discussed further above or other devices.

The electronic system 1000 comprises an electronic control unit 1100. The control unit may comprise the controller 700 as discussed further above. Specifically, the control unit may comprise a processor arrangement 23 as discussed further above. Also, the control unit 1100 may comprise one, or a plurality of memory units, such as program memory 25 and main memory 24 discussed further above in conjunction with FIG. 7. The control unit 1100 is expediently designed to control operation of the electronic system 1000. The control unit 1100 may communicate via wired interfaces or wireless interfaces with further units of the electronic system 1000. It may transmit signals containing commands and/or data to the units and/or receive signals and/or data from the respective unit. The connections between the units and the electronic control unit are symbolized by the lines in FIG. 20. However, there also may be connections between the units which are not illustrated explicitly. The control unit may be arranged on a conductor carrier, e.g. a (printed) circuit board. The other unit(s) of the electronic system may comprise one or more components which are arranged on the conductor carrier as well.

Electronic system 1000 may further comprise a motion sensing unit 1200. The motion sensing unit 1200 may comprise one or a plurality of sensors, e.g. the sensors 215a and 215b described further above. In case optoelectronic sensors which detect electromagnetic radiation, such as IR sensors, are used, the motion sensing unit may additionally comprise a radiation emitter which emits the radiation to be detected by the sensor. However, it should be noted that other sensor systems, e.g. magnetic sensors could be employed as well. A motion sensing unit which has an electrically operated sensor and an electrically operated source for stimulating the sensor—such as a radiation emitter and an associated sensor—the power consumption may be particularly high and, hence, power management may have particular impact. Each sensor may have an associated radiation emitter. Motion sensing unit 1200 may be designed to detect and preferably measure relative movement of two movable members of a dose setting and drive mechanism of or for the drug delivery device during a dose setting operation and/or during a dose dispensing operation. For example, the motion sensing unit may measure or detect relative rotational movement of two movable members of the dose setting and drive mechanism with respect to one another. Based on movement data received from or calculated from the signals of the unit 1200, the control unit may calculate the dose data.

Electronic system 1000 may further comprise a use detection unit 1300. The use detection unit may be associated with the user interface member or members such that manipulation of the member for setting and/or delivering a dose thereof may be detected. When the manipulation is detected, the use detection unit generates or triggers generation of a use signal. The use signal can be transmitted to the electronic control unit 1100. The electronic control unit may, in response to the signal, issue a command or signal to one of, an arbitrarily selected plurality of, or all of the other electrically operated units of the system. For example, the control unit may cause that the respective unit is switched from a first state, e.g. a sleeping state or idle state with a lower power consumption or an off state with no power consumption, to a second state with an increased power consumption. The switching may be done by an according switching command or signal issued by the electronic control unit to the respective unit. In response to the use signal all units may be switched to the second state or just selected units. If only selected units are switched to the second state with higher power consumption, it is expedient that these units are intended to be used during the operation which is intended to be commenced by the user or which has been commenced. For example, typical times required to switch the motion sensing unit to the second state are between 2.5 ms and 3.2 ms, e.g. after generation of the use signal or actuation of the user interface member to initiate the operation of the system, e.g. the dose delivery operation.

The electronic system 1000 may further comprise a communication unit 1400, e.g. an RF, WiFi and/or Bluetooth unit. The communication unit may be provided as a communication interface between the system or the drug delivery device and the exterior, such as other electronic devices, e.g. mobile phones, personal computers, laptops and so on. For example, dose data may be transmitted by the communication unit to the external device. The dose data may be used for a dose log or dose history established in the external device. The communication unit may be provided for wireless or wired communication.

Electronic system 1000 may further comprise an electrical power supply 1500, such as a rechargeable or non-rechargeable battery. The power supply 1500 may provide electrical power to the respective units of the electronic system.

When the system is in the first state, e.g. with neither the motion sensing unit being active nor the communication unit, the current consumption may be 200 nA. When (only) the motion sensing unit is active, the power consumption may be 0.85 mA. When the communication unit is active, e.g. in addition to the motion sensing unit or only the communication unit, the power consumption may be 1.85 mA.

Although not explicitly depicted, the electronic system may comprise a, preferably permanent and/or non-volatile, storage or memory unit, which may store data related to the operation of the drug delivery device such as dose history data, for example.

In one embodiment, the electronic control unit 1100 may be configured to reduce the power consumption of the respective unit, i.e. to switch the unit back to the first state. This is suitable, for example if an event which is relevant for that unit, e.g. a motion sensing event for the motion sensing unit, has not occurred in a predetermined time interval after the unit has been switched from the first state into the second state and/or after the use signal has been generated. The monitoring of the time interval may be achieved by a timer unit which is operatively connected to the electronic control unit (not explicitly shown). In case, after the use signal, there is no signal generated by the motion sensing unit within the predetermined time interval, the entire system may be switched again to the first state. This time interval may be greater than or equal to one of the following values 0.2 s, 0.5 s, 1 s, 5 s, 10 s, 15 s, 20 s, 25 s, 30 s.

It goes without saying that the electronic system 1000 may comprise further electronic units other than the ones shown such as other sensing units, which sense or detect different quantities or events than the relative movements which the motion sensing unit detects.

The respective unit may be integrated into a user interface member 1600, e.g. a dose setting and/or delivery button (e.g. knob 12 of the device discussed previously) of the electronic system.

The use detection unit 1300, when it is operational to generate the use signal, e.g. the setting signal and/or the delivery signal, expediently has a lower power consumption than the motion sensing unit 1200 when it is active.

When investigating the operation of the trigger switch arrangement with switch 800 which has been discussed further above, it has been noticed that designing such a trigger switch arrangement is challenging, e.g. as an axial movement is used to trigger the switch. For example, it needs to be ensured that the switch is triggered before the delivery operation is commenced, e.g. before a clutch is disengaged. Furthermore, the switch may be subject to considerable axial displacement as the clutch needs to be disengaged by relative axial movement after the switch has been triggered, which may put considerable load onto the switch. These considerations render the design of an axial trigger switch pretty complex and, potentially, unreliable.

In the following, a couple of embodiments are discussed which may address drawbacks of existing systems. Also, it may be desirable to provide further embodiments of systems which are configured to wake up further electronic components of the systems (only) when the operation of the respective component is needed. In general, systems which have a configuration to allow one or more further electronic components to be woken from a low power state or sleeping state will be discussed below.

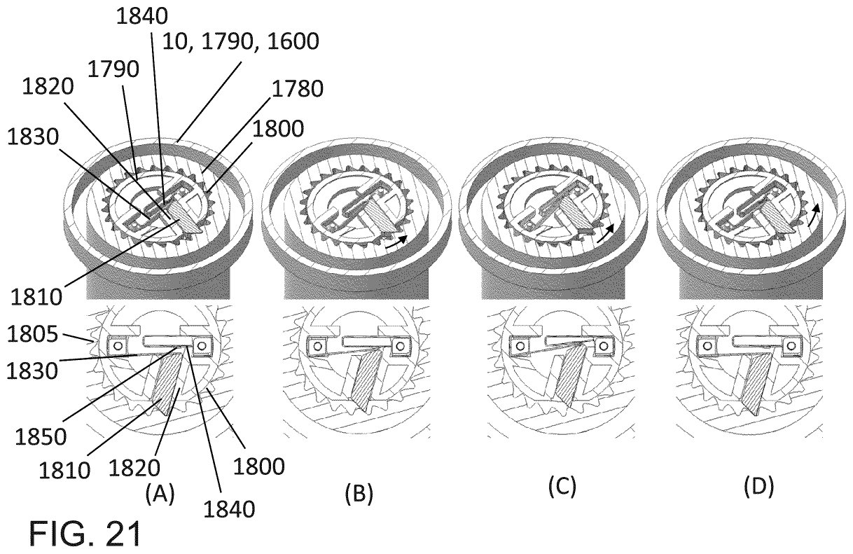

FIG. 21 illustrates an embodiment of an electronic system on the basis of four different representations A through D during operation of the device utilizing the electronic system. In this embodiment, when the use signal is generated it is ascertained that the respective operation—in this example the dose delivery operation but likewise possible for the dose setting operation—has already been initiated or commenced. This is advantageous over triggering the wake-up process for the motion sensing unit via axial movement of the user interface member, e.g. an injection button. The axial trigger movement of the user interface member could happen inadvertently e.g. by parts in a handbag hitting and moving the button while the user is walking around, whereas the relative movement which is required for actually performing the desired operation is less likely to occur inadvertently. The present embodiment uses a relative movement of two members occurring during the dose delivery operation to switch the electronic system from the first state of lower power consumption to the second state of higher power consumption where, for example, the motion sensing unit and/or the communication unit are powered up. Integrating the switching process into the dose delivery operation has the advantage that, in case the currently dispensed dose should be monitored via the motion sensing unit, the activation of the motion sensing unit may occur immediately when it is needed—actually only after when the operation it should monitor has been commenced already. It should be appreciated that, of course, similar switching operations or the same switching operations can also be performed during the dose setting operation. However, the following description focuses on the dose delivery operation. FIG. 21, in the upper portion, shows a perspective view on members of the electronic system relevant in connection with the disclosed concept and the lower portion shows a top view onto these members. The different representations A through D illustrate different relative positions of the members during the dose delivery operation.

Representation A illustrates the situation when a dose has been set and before the dose delivery operation is commenced. Depicted are schematical representations of a housing 10, a first member 1780 and a second member 1790. Instead of a portion of the housing 10, the outer portion may also be a portion of the first member and/or a portion of the user interface member 1600. The first member and the second member are part of a dose setting and drive mechanism of or for the drug delivery device. For example, the first member 1780 may be the dial sleeve or number sleeve or a member axially and/or rotationally locked thereto and the second member 1790 may be the drive sleeve or the user interface member, e.g. a dose and/or injection button. At least a section of the second member 1790 may be received in the first member 1780.

For dose delivery, the first member 1780 moves, e.g. rotates, relative to the second member 1790. The second member may be directly or indirectly engaged with a piston rod to drive the dose delivery operation. During dose setting, the first member 1780 and the second member 1790 may co-rotate relative to the housing 10. Thus, these members may be rotationally locked relative to one another such as by a clutch interface which is established during dose setting (e.g. via mating teeth on the two members, not shown). During and/or for dose delivery, the clutch interface may be released. The length of the device may be increased by an amount which is proportional to the size of the set dose by the first member and/or the second member protruding from the housing during the dose setting procedure. However, the disclosed concept would also work without such a dial extension during dose setting. During dose setting, a dose setting member, e.g. the user interface member 1600 which may be integrated into the second member may be rotated relative to the housing 10 in an incremented manner, e.g. in whole-number multiples of a unit setting increment. This may be achieved via a dose setting interface, e.g. a ratchet interface (not explicitly shown), which is pitched in accordance with the unit setting increment. Such an interface may be formed between the first member 1780 and the housing or another member which is stationary during the dose setting procedure and a member movable, e.g. rotatable, relative to that member stationary during setting. The dose setting interface may define stable positions of the respective member with respect to the housing which are separated in the angular direction by one unit setting increment. For example, one unit setting increment may correspond to a rotation angle of 15°. Thus, two stable rotational positions may be separated by 15° in the angular direction.

In order to switch the electronic system from the first state into the second state, this concept utilizes a use signal generation interface to generate a use signal before the first member and the second member have rotated relative to one another by one unit increment, which will be discussed further below. The use signal generation interface is also an incremented interface and, preferably, is incremented or pitched in the same manner as the dose setting interface or may have even finer pitch or smaller increments. In the depicted embodiment, the first member 1780 is provided with circumferentially disposed ratchet pockets 1800. Two adjacent ratchet pockets are separated by a ratchet tooth 1805. The respective teeth 1805 are circumferentially disposed. In the depicted embodiment, the ratchet pockets 1800 define 24 stable positions distributed over 360°, i.e. two stable positions are separated by an angle 15°. The respective ratchet pocket or the teeth 1805 defining the pockets may be radially oriented, i.e. the teeth may have a radial free end. The teeth may point inwardly. Alternatively to the first member comprising the ratchet, a use signal generation interface member 1880 may be provided with the ratchet, which is at least rotationally locked to the first member 1780, which may be the dial sleeve or number sleeve, for example. In the depicted embodiment, two adjacent ratchet pockets are delimited by differently sloped surfaces, a steep surface and the less steep surface.

This ratchet interface is provided to define a unidirectional interface where relative rotation between the two components is only possible in one rotational direction. Thus, the interface can be used to block rotation in the other direction which may increase the safety of the device employing the system. In the present configuration, rotation of the first member 1780 with respect to the second member 1790 in the counterclockwise direction is allowed. Expediently, the allowed rotation is the one which occurs during the dose delivery operation. Thus, the clutch interface has to be released before the system is switched into the second state and also a part of the dose delivery operation has to be performed already.

Moreover the system comprises a switching feature 1810, e.g. a pin-like component. The switching feature 1810 is movable, e.g. radially, relative to the ratchet pockets 1800 in response to rotation of the first member 1780 relative to the second member 1790. The switching feature expediently shuttles between two different positions. In a first position, e.g. a radially outward position, the feature engages the ratchet pocket 1800 and in a second position, e.g. an inward position, it engages an end surface of the ratchet tooth. If the first member 1780 is rotated relative to the second member 1790, on account of the sloped surface delimiting the ratchet pocket in the angular direction which is opposite to the rotational direction, the switching feature 1810 is inwardly displaced, e.g. radially inwardly, from the first position to the second position. The switching feature 1810 may be guided such that, in response to rotation of the first member 1780 it moves only linearly, e.g. in the radial direction or predominantly in the radial direction. For this purpose, a guide slot 1820, e.g. a linear slot, is provided in the second member 1790. A free end of the switching feature may have a shape complementing the ratchet pockets. The switching feature 1810 may be tightly received in the respective ratchet pocket 1800. The switching feature 1810 may be rotationally locked to the second member 1790. The switching feature may be radially oriented or be arranged such that it defines an angle with the radial direction. The switching feature may be electrically insulating, e.g. of plastic.

The system further comprises a first electrical contact feature 1830, e.g. an elastically displaceable feature. The feature 1830 may be a metal feature and/or a contact strip. The system further comprises a second electrical contact feature 1840, e.g. an elastically displaceable feature. The feature 1840 may be a metal feature and/or a contact strip. The respective contact feature may be fixed to the second member 1790. The respective feature may have a free end and/or a portion which is displaceable relative to the portion where the respective feature is fixed to the second member 1790. The first contact feature 1813 is arranged to be moved towards the second feature 1840 when the switching feature 1810 is displaced inwardly.

In the situation depicted in representation A the first electrical contact feature 1810 may urge the switching feature 1810 outwardly to maintain the switching feature in engagement with the ratchet pocket 1800 which it is currently engaged with. Thus, in the representation A, the switching feature 1810 and the first electrical contact feature abut. The contact features 1840 and 1830, however, are separated and not conductively connected.

When, starting from the situation in representation A, the dose dispensing operation is commenced which involves relative rotation of the first member with respect to the second member, the switching feature 1810 will be displaced inwardly. The switching feature 1810 carries with it the first electrical contact feature 1830 and moves it towards a portion of the second electrical contact feature 1840 until the contract features are in mechanical and electrical contact with one another as depicted in representation B. By way of this contact, a use signal may be generated which can be used to trigger the switching of the electronic system into the second state of higher power consumption as has been discussed above already. For example both contact features may be conductively connected to the power supply 1500 and, current can only flow to provide the use signal if the contact features are conductively connected. The use signal is preferably generated when the first member has been rotated relative to the second member by less than one unit increment as depicted in representation B. Preferably, a piston rod of the dose setting and drive mechanism has been displaced already to begin dispense of a first unit increment of drug from a container of the device before the use signal has been generated, e.g. driven by user force transferred to the piston rod from the user via the second member 1790. The portions of the first and second contact feature 1830 and 1840 which abut are displaced together by the switching feature 1810 as is depicted in representation C. This ensures that the use signal is generated in all tolerance conditions. When the rotation is continued, the switching feature 1810 is again allowed to move outwardly into engagement with the next ratchet pocket 1800. This movement is driven by the resilient force provided by the first contact feature 1830. Thus, after the rotation has been completed by one unit increment (signal generation increment), the switching feature 1810 again rests in a ratchet pocket. Also, the second contact feature 1840 returns to its initial position such that, after the rotation by one unit increment has been completed, the system is in the situation depicted in representation D which, aside from the relative rotation by one unit increment, corresponds to the situation in representation A. When dose delivery continues, the features are brought into contact again during the next increment, where a signal is generated again.

Accordingly, the use signal may be generated in an incremented fashion depending on the relative rotation between the two members 1780 and 1790. Also, it is ensured that the use signal has been generated before the rotation by one unit increment has been completed. Thus, if the motion sensing unit 1200 has been switched to a sensing state where the components of the motion sensing units, such as LED(s) and sensor(s), are supplied with electrical power and detects the relative rotation between the first and second members during the further course of the dose delivery operation, the offset by one unit increment may be taken into account, e.g. by adding the angle corresponding to one unit increment of the use signal generation interface to the result of the calculation of a rotation angle derived from the signal of the motion sensing unit. It is advantageous that the dose setting interface and the use signal generation interface are incremented in the same manner as has already been discussed above. However, different increments are also possible where the use signal generation interface is preferably finer pitched, i.e. with smaller increments in the angular direction, than the dose setting interface. It is, however, also possible to have a coarser pitch in the increments of the use signal generation interface than in the ones of the dose setting interface.

In the portions, where the first electrical contact feature and the second electrical contact feature are designed to abut to trigger the generation of the use signal, a protrusion or bulge may be arranged, which points from one of the portions towards the other one of the portions. In the depicted embodiment such a protrusion 1850 is provided on the second electrical contact feature 1840. This may also assist to guarantee a use signal even in extreme tolerance condition. However, such a protrusion need not be present. Alternatively, both contact features could be provided with such a protrusion 1850. Moreover, it is also conceivable to use a separate biasing member to drive the respective movement of the switching feature and/or any movement of the respective contact feature, wherein the biasing member is biased while the contact features are in abutment and/or moved together.

Having radially oriented ratchet features (pockets and teeth) and the radially/transversely oriented switching feature facilitates an axial relative movement between the first member and the second member which may be necessary to release/establish a clutch interface and/or in order to switch between the dose setting operation and the dose delivery operation. However, there may also be systems which do not involve such a switching of a clutch interface. For allowing the relative axial movement, the ratchet pockets may have an axial extension which is sufficient to permit this movement. That is to say, the switching feature may be axially guided within the ratchet pocket by a distance which corresponds to the distance which is required to switch the state of the clutch interface, e.g. from a coupled or established state to an uncoupled or released state or vice versa.

It is advantageous, if the force required to operate the switch which is established by the first electrical contact feature and the second electrical contact feature is as low as possible in order to avoid that the user has to exert too much force during the dose delivery operation.

In the depicted situation, use signals are generated throughout the entire dose delivery operation every time the switching feature 1810 is displaced inwardly when it changes position from one ratchet pocket into the next ratchet pocket.

In one embodiment the first member 1780 or the use signal generation interface member 1880 may be axially coupled, e.g. locked relative to the second member 1790, e.g. the dose button and/or drive sleeve. This configuration avoids relative axial movement of the member with the ratchet features (1800, 1805) relative to the member the rotation of which should be monitored and/or the switching feature 1810. In this case, the ratchet may be axially displaced relative to the member the rotation of which should be monitored. The relative axial position of switching feature 1810 and ratchet may be constant. Alternatively, the switching feature is axially displaced relative to the ratchet when the dose delivery operation is being initiated or commenced.

Apart from generating one or more use signals, the disclosed configuration, may be used in order to generate an audible and/or tactile feedback during the dispensing operation and/or to prevent relative rotation between the first and second members in an undesired direction.

In the present configuration, the generation of the use signal may be triggered without a direct contact from a user, i.e. by a switch which does not have to be mechanically contacted by the user. Moreover, the event which causes the generation of the use signal is integrated into the regular operation procedure of the drug delivery device, particularly into the dose delivery operation after the user interface member/button has been moved into the position required for this operation and when the user interface member has reached that position, the relative movement between first and second member has been commenced. A separate user action is not necessary in order to switch the electronic system to the second state. Moreover the use signal increments can be matched to the dose unit increments which is advantageous, as the switching of the state of the electronic system can be precisely tuned to the dose unit increments.

In an alternative design, the switching feature may be pivotally mounted to the first member such that rotation of the first member causes a pivoting movement in a plane perpendicular to the rotation axis.

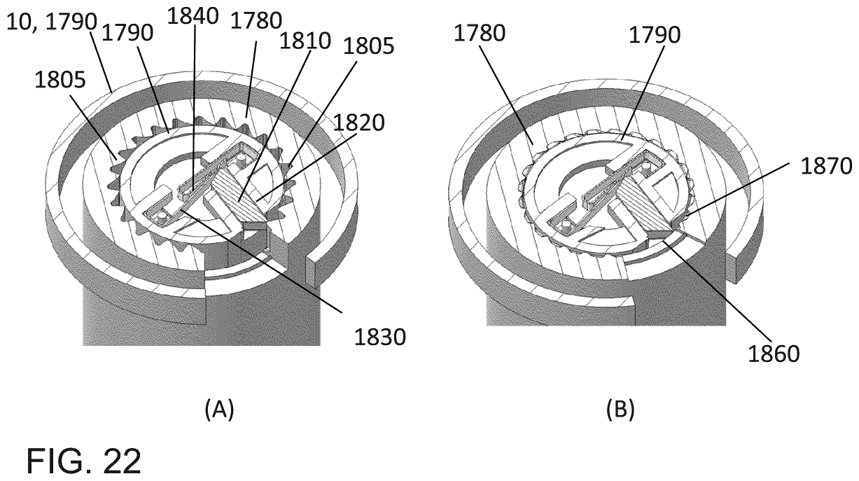

FIG. 22 illustrates another embodiment of an electronic system on the basis of two representations A and B. As will be easily acknowledged, this embodiment is very similar to the one discussed in conjunction with FIG. 21. However, in this embodiment, the situation is a little different as regards engagement of the switching feature 1810 with the ratchet, which is defined by the ratchet pockets 1800 which are separated by ratchet teeth 1805. As has been discussed further above, in conjunction with the embodiment in FIG. 21, the switching feature 1810 may be engaged with the ratchet pockets and/or the ratchet teeth in a first axial relative position of the first member 1780 and the second member 1790 and in a second axial position of that movable members. The first and the second axial position may be the two extreme positions the first and second member may have relative to one another. For example, starting from an initial position the second member 1790 may be moved axially, e.g. distally, relative to the first member 1780 until it has reached an end position. Therefore, in FIG. 21, in the end position, the switching feature 1810 is also engaged with the ratchet teeth and/or the ratchet pockets. As there is relative rotational movement between the first member 1780 and the second member 1790 in the dose delivery operation, force or torque needs to be provided to continuously increment the ratchet, e.g. by the user when the device is user driven. However, after the initial use signal generation, a further use signal may not be necessary anymore as the electronic system has been switched to the state of higher power consumption already such that the dose recording via the dose recording or motion sensing unit can be performed. Consequently, it may be advantageous to reduce the force. Therefore, in this embodiment, in the first relative axial position of the first member 1780 and the second member 1790, the switching feature 1810 engages the ratchet and relative rotation between the first and the second member results in the switching feature being displaced to generate the use signal. However, when the first and the second members 1780 and 1790 have reached the second relative axial position, the switching feature 1810 has disengaged the ratchet. The use signal is still generated by the relative rotation of the first member and the second member. The first relative axial position may be the regular position of the two members, e.g. maintained by a biasing member such as the clutch spring. When in the second relative position a counterforce, e.g. by the user, is removed the first relative position may be established again by the spring.

Representation A illustrates an intermediate state during the relative movement between the first axial position and the second axial position of the members 1780, 1790 when the generation of the use signal has already been triggered (indicated by the abutting contact features 1830 and 1840). Specifically, the state in representation A in FIG. 22 corresponds to the state in representation C of FIG. 21. However, at the end of the relative axial movement the switching feature 1810 has disengaged the ratchet. For example, it may be axially offset from the ratchet, e.g. distally offset. This situation is shown in representation B of FIG. 22.

Here, the second relative axial position between the first member 1780 and the second member 1790 is illustrated. As is immediately apparent, the switching feature 1810 now engages, in particular radially, a surface 1860. The surface may be smooth, in particular as compared to that area where the ratchet pockets 1800 and the ratchet teeth 1805 are disposed. The surface 1860 may be cylindrical. Axially between the surface 1860 and the respective ratchet pockets 1800 or ratchet teeth 1805 a ramp 1870 is arranged. The ramp may be defined in an end section of the respective ratchet pocket 1800 which engages the switching feature 1810 during the relative movement of the first member and the second member from the first axial position into the second axial position. The ramp may be inclined in the axial direction, e.g. inwardly, when seen along the movement direction of the switching feature 1810 relative to the ratchet pocket 1800 to disengage switching feature 1810 from the pocket. When engaged by the switching feature 1810, the ramp may guide the switching feature 1810 in the radially inward direction until the ratchet pocket 1800 is disengaged. Then the switching feature 1810 may engage the surface 1860. The surface 1860 may react the residual force provided by the elastically deformed first and/or second contact feature(s) 1830, 1840. Accordingly, in the second relative axial position between the first and second members, the contacts may be continuously closed and a use signal may be generated or otherwise power may be drained from the power supply.

It is not necessary that the contact features 1830 and 1840 engage one another in the second relative axial position of the first and second members. This can be achieved by widening the interior of the system, e.g. of the first member 1780, in the region where the switching feature 1810 is disposed in the second relative axial position of the first and second members. This is not explicitly illustrated. In this case, a ramp which is inclined outwardly when seen along the movement direction, may be axially disposed between the ratchet pockets 1800 and the switching feature 1810. This ramp may serve to reengage the switching feature with the ratchet pockets 1800 when the first relative position between the first and second member is reestablished after the delivery operation has been completed. Here, the radial width in that portion of the first member, where the switching feature is axially arranged in the second relative axial position of the first member and the second member, is preferably greater than the radial width defined between the free ends of the ratchet teeth.

In this embodiment, the axial extension of the ratchet pockets and/or teeth may be greater than the distance required to release the clutch interface for rotationally decoupling the first member and the second member from each other. The axial extension of the ratchet pockets is expediently chosen such that it permits triggering of the use signal in even extreme tolerance conditions. The user interface member/button may be movable in the distal direction from an initial position to a dose delivery position. The distance may be greater than required to release the clutch interface.

In this embodiment, in case the contact features are in contact in the second relative axial position (as in representation B of FIG. 22), the user interface member could be used to generate the use signal without relative rotation being required as the contact is closed in the second position. Therefore, in terms of power drainage, the option where the contact is not closed in the second relative axial position may be advantageous.

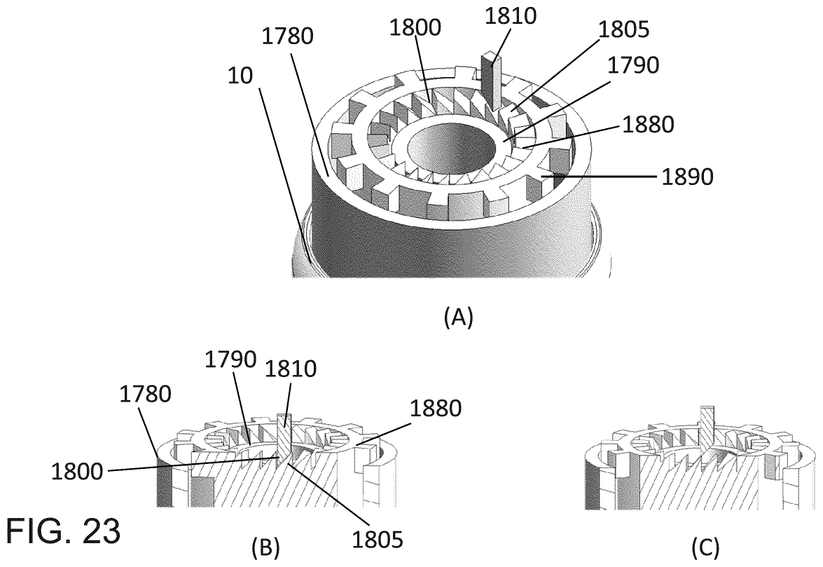

FIG. 23 illustrates yet another embodiment of an electronic system on the basis of three different representations A through C. The general setting of the system is very similar to the systems disclosed further above in conjunction with FIGS. 21 and 22. Therefore, the following discussion will focus on the differences.

As in the previous embodiments, the first member 1780, e.g. the number sleeve, the dial sleeve and/or a component rotationally and/or axially attached thereto, and the second member 1790, e.g. a drive sleeve or a dose and/or injection button, are provided. The housing 10 is also depicted. The first member and the second member may be rotationally locked to one another during the dose setting operation but there is relative rotation during the dose delivery operation. For example, the first member 1780 may rotate relative to the second member 1790 and relative to the housing 10 during dose delivery. The second member 1790 may be rotationally locked to the housing during dose delivery. The angle of rotation of the first member relative to the second member may, as in the embodiments which have been discussed already, be indicative for the size of the dose which has been dispensed already.

In this embodiment, again, relative rotation between the first and second members 1780, 1790 is used to trigger the use signal generation. As in the embodiments described further above, for this purpose, the switching feature 1810 is provided. The switching feature 1810 in general is configured in the same way as discussed previously. However, its orientation is different, as it is axially oriented as can be seen in representation A which presents an oblique view onto the described elements of the system which, of course, may have additional elements. The switching feature 1810 moves axially in order to generate the use signal by triggering a sensor or switch which, contrary to the embodiments discussed previously is not explicitly illustrated. The sensor or switch could be implemented via electrically contacting features or via a force sensor or pressure sensor which is mechanically contacted by the switching feature in order to trigger the generation of the use signal. The use signal may be fed to the electronic control unit and may be used to trigger the switching of the system into the second state by the control unit.

The switching feature 1810 is again operatively coupled or engaged with ratchet pockets 1800 and/or ratchet teeth 1805. The ratchet teeth and pockets together define a ratchet. Contrary to the previous embodiments the ratchet pockets 1800 and/or the ratchet teeth are axially oriented. For example, free ends of the ratchet teeth 1805 may be oriented in the axial direction such as in the proximal direction. The ratchet teeth may have a helical configuration. That is to say an inclined side face of a tooth may extend helically when viewed in top view along the rotation axis, e.g. in the distal direction. When there is relative rotation between the ratchet teeth 1805 and the switching feature 1810, the switching feature may be displaced axially relative to the ratchet pocket and/or the ratchet teeth, e.g. away from the teeth and/or outwardly relative to the ratchet pockets. This axial movement may be used to trigger the use signal.

In the present embodiment, contrary to the preceding embodiments, a (separate) signal generation interface member 1880 is explicitly illustrated. Such a member may be provided in the preceding embodiments as well or may be dispensed with. The signal generation interface member 1880 or at least a portion thereof may be arranged radially or axially between the first member 1780 and the second member 1790. The ratchet may be provided on the signal generation interface member 1880. The signal generation interface member 1880 may be mechanically coupled to the first member 1780 and/or the second member 1790. For example, the signal generation interface member 1880 may be rotationally locked to that member which rotates relative to the housing and relative to the other member. For example, it may be rotationally locked to the first member 1780. The signal generation interface member may be axially locked or coupled to the other member which, preferably does not rotate relative to the housing 10, e.g. during dose delivery. For example, the member 1880 may be axially coupled to the second member 1790. The one of the first and the second member to which the signal generation interface member 1880, which may have a ring-like configuration, is axially secured, locked or coupled may be the member which carries the sensor or switch which is triggered by the movement of the switching feature 1810. When axially coupled to one of the members, e.g. the second member 1790, the signal generation interface member 1880 may follow axial movement of that member, especially in the distal direction and/or in the proximal direction.

When the signal generation interface member 1880 is rotationally locked to the other one of the members, e.g. the first member 1780, it may rotate together with that member relative to the housing and/or relative to the other member, e.g. relative to the second member 1790.

In the depicted embodiment, the signal generation interface member 1880 is preferably rotationally locked to the first member 1780 and may be axially locked to the second member 1790. Accordingly, when a clutch interface between the first member and the second member is released, e.g. by displacing the second member 1790 axially, e.g. distally, relative to the first member 1780, the rotational locking of member 1880 to the first member 1780 may be maintained and the signal generation interface member is moved together with the second member relative to the first member in the axial direction. The rotational locking may be effected by splines which provide a rotational lock between interface member 1880 and first member 1780 (not explicitly illustrated). The axial lock may be effected by a circumferential groove in the second member 1790.

The first member 1780 or the signal generation interface member 1880 may be provided with encoder surfaces or detection regions 1890 for the motion sensing unit such as the reflecting surfaces are regions 70a which have been described previously. The regions between the detection regions may be non-reflective regions 70b. The detection regions 1890 in the depicted embodiment are formed by protrusions protruding radially outwards where other configurations are possible. The regions are disposed circumferentially, preferably evenly, and/or aligned axially, i.e. arranged at the same axial position, where the axis is expediently the longitudinal axis of the system or the rotation axis of the first member 1780 relative to the housing 10. In other words, the regions 1890 may be evenly distributed. All regions 1890 may have the same configuration e.g. angular width and/or shape.

Representations B and C again show different stages during the relative movement of the first member 1780 relative to the second member 1790. As will be easily acknowledged, when the first member 1780 rotates, particularly in the clockwise direction, the switching feature 1810 will be displaced axially out of the ratchet pocket 1800 it currently resides in representation B on account of the interaction with the inclined side surface of the ratchet tooth 1805 until it disengages the ratchet pocket 1800 (see representation C). As rotation continues, the switching feature 1810 reengages the subsequent ratchet pocket 1800, e.g. on account of a biasing member and/or an elastically deformable contact feature (not explicitly shown), which biases the switching feature into engagement with the ratchet pockets 1800, e.g. by a biasing member which provides an axially, e.g. distally, directed force onto the switching feature 1810. In the situation in representation C the use signal is generated, e.g. by the feature 1810 triggering a switch such as a micro switch. Preferably, the operating force of the switch is small to keep the total increase in force required to drive system at a reasonable level. When from representation C the rotation is continued, the subsequent ratchet pocket is engaged by the switching feature.

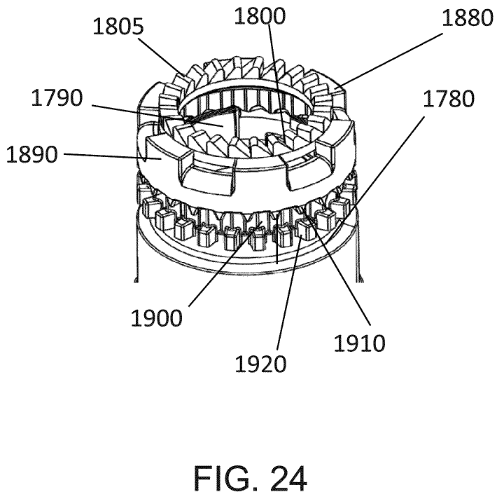

FIG. 24 illustrates another embodiment of the electronic system. As in the previously described embodiments, a use signal generation interface member 1880 is provided. This member 1880 comprises ratchet pockets 1800 and the ratchet teeth 1805 which define the incremented interface which generates use signals in cooperation with the switching feature 1810 as discussed previously already. Adjacent teeth may be separated by an angle corresponding to one unit setting increment. Thus, the unit setting increment may be equal to the use signal generation increment as has been discussed further above. Therefore, the unit setting increment is used as reference below for some angles. However, it should be noted that the use signal generation increment could be used as well, in case it is different from the unit setting increment.

The use signal generation interface member 1880 is expediently rotationally secured to the first member 1780 and axially secured relative to the second member 1790 as has been discussed already further above. The interface which rotationally secures the member 1880 to the first member 1780 is realized by spline features 1900, e.g. axially oriented ribs. The spline features 1900 may be provided on the first member 1780. Corresponding features on the use signal generation interface member 1880 may be arranged to engage the spline features 1900. Of course, the position of the spline features and the corresponding features which engage the spline features could also be reversed such that the spline features are provided on member 1880 and the corresponding features provided on the first member 1780. The use signal generation interface member may engage the first member 1780 in a proximal end region thereof. The proximal end region of the first member 1780 may be received within the use signal generation interface member 1880.

The use signal generation interface member 1880 is axially movable relative to the first member 1780 between a first position and a second position, where the first position is shown in FIG. 24. The member 1880 may be rotationally locked relative to the first member 1780 in both positions or in just one of these position. The second position may be distally offset relative to the first position, where the distal direction, in FIG. 24 is the downward direction. In the second position, axially oriented engagement features 1910 of the use signal generation interface member may engage corresponding features 1920, e.g. slots, in the first member 1780. The engagement may either establish the rotational lock between the first member and the use signal generation interface member or may stabilize the relative angular position between the first member 1780 and the use signal generation interface member 1880. This may increase the accuracy of the motion sensing and/or the determination of the delivered dose as the rotational orientation between the first member, which rotates in the dose delivery operation and of which rotation should be monitored or measured via the motion sensing unit, and the use signal generation interface member 1880, which may carry the detection regions 1890, which are used to monitor the rotational or angular position of the first member in the dose delivery operation, is stabilized. The interface formed during the movement of the use signal generation interface member 1880 from the first position into the second position may be self-centering, which may be achieved by slanted surfaces of one of the engagement features 1910 and 1920. In the depicted embodiment, the features 1910, which may be pointed teeth, have slanted surfaces.

The angular distribution of the engagement features 1910 and/or 1920 may have a pitch which is determined by the angle corresponding to one use signal generation increment and/or one unit setting increment, where, as already discussed, the angles corresponding to one use signal generation increment and one unit setting increment may be identical. The distance by which the use signal generation interface member is moved axially relative to the first member 1780 may be determined by the clutch release distance, e.g. d_{c}, which has been discussed further above already.

In the depicted embodiment the use signal generation interface member 1880 provides the use signal generation interface by ratchet teeth and ratchet pockets and also the detection regions 1890 for determining the amount of relative rotation during dose delivery. This is advantageous from a manufacturing perspective as a structure for generating the use signal as well as a structure for monitoring movement can be integrated into one component, which can be integrated into a device more easily than separate components. The detection regions 1890 may be provided on an outer surface and the use signal generation interface may be provided on the inner surface of the member 1880.

Of course, this embodiment could be combined with any one of the remaining embodiments set forth in this disclosure.

In the embodiments which have been discussed previously, in particular in conjunction with FIGS. 21 to 24, the use signal was generated and/or the motion sensing unit has been activated (rendered operative) when the relative movement between the two members required for the dose delivery operation has already been initiated, e.g. with the piston rod moving already and/or drug being expelled from the device. Thus, the use signal is generated right when or, in fact, closely after the motion sensing unit 1200 is needed. This is advantageous in terms of minimizing unnecessary power consumption, e.g. due to the motion sensing unit being active although no dose delivery operation is performed after its activation. However, the dose information which can be calculated, e.g. in the electronic control unit, from the movement of the first member relative to the second member during the dose delivery operation may be inaccurate, if the (potential or deliberate) “late activation” of the motion sensing unit is not taken into account at all. For the use signal generation, a relative rotation by up to one unit setting increment or one use signal generation increment (these two increments may be equal) may be required as has been discussed further above.

As the motion sensing unit is still inactive during the movement required to render the motion sensing unit operative, the positional change which cannot be detected can be taken into account by adding a constant value, e.g. corresponding to one unit setting increment, by default to the quantity of the delivered dose which is determined by the motion sensing unit. However, it will be appreciated that it may take some time until the motion sensing unit will indeed be powered up from the generation of the use signal, e.g. due to the transmission of the use signal to the electronic control unit, the required processing of the use signal in the electronic control unit and/or the transmission of an activation signal from the electronic control unit to the motion sensing unit. Furthermore, the positional change may depend on the rotational speed and/or the acceleration occurring during the dose delivery operation. The operation may be user driven. Accordingly there may be a wide range of potential speeds and accelerations. Therefore, it is possible that the motion sensing unit is not active when the second unit increment of the set dose was delivered in the delivery operation or activated only late during the delivery of the second unit increment. This would result in an inaccurately determined delivered dose by the motion sensing unit as it is unsure how much of the set dose has already been dispensed when the motion sensing unit has been activated, e.g. one unit increment or a fraction thereof or more than one unit increment, e.g. two unit increments. Thus, the approach of adding a constant value to the determined dose information may not yield an accurate result.

The faster the rotation, the more likely it is that more than the constant value has already been delivered before the motion sensing unit has been activated. Especially, activating the motion sensing unit 1200 may not only require the electronic control unit 1100 to issue a command to the motion sensing for activating it but rather, alternatively or additionally to the motion sensing unit activation, may require activation of the electronic control unit itself, e.g. the microprocessor or microcontroller. Activating the electronic control unit—in particular such that the electronic control unit can then activate the motion sensing unit—may require loading an integrated or embedded operating system (e.g. software-based), which, aside from supplying electrically powered components with appropriate power from the power source, will also require some time.

Typical minimum time spans which are required for activating one of the units, e.g. the motion sensing unit or the electronic control unit, may be greater than or equal to one of the following values: 0.1 ms, 0.2 ms, 0.3 ms, 0.4 ms, 0.5 ms, 1 ms, 2 ms (ms: milliseconds). Alternatively or additionally, the time span may be less than or equal to one of the following values: 5 ms, 4 ms, 3 ms. For example, the time spans for activating one unit may be between 0.1 ms and 5 ms. If two units are activated sequentially, such as the electronic control unit, e.g. in response to the use signal, and the motion sensing unit, e.g. by an activation signal generated by the electronic control unit and transmitted to the motion sensing unit after activation of the electronic control unit has been completed, this may need more time, e.g. twice a typical time span for activating one unit. The time required for activating the electronic control unit may be greater than the time for activating the motion sensing unit. For example, before the first information can be retrieved from the motion sensing unit, 2.5 ms may have passed already.

As the use signal to wake up the motion sensing unit and/or the electronic control unit in the embodiments discussed further above is only generated after the dose delivery operation has already been commenced, the likelihood that one or even more unit setting increments are missed is increased, because the time available for powering up the relevant unit(s) of the electronic system is reduced. For example, two unit setting increments may be missed by the motion sensing unit detecting or reading movement of the encoder component (e.g. the use signal generation interface member 1880 or a separate encoder component).

In order to increase the accuracy of the calculation of dose information from the motion sensing unit (regardless of whether it is activated before the dose delivery operation is commenced or thereafter) it is proposed herein that positional information on the position which the encoder component has relative to the motion sensing unit before the dose delivery operation is commenced is stored in the system, e.g. in a non-transitory memory of the system. The stored positional information or data can then be compared with positional information or data derived from the first data signal or reading signal generated by the motion sensing unit. If the stored positional information and the positional information derived from the first data signal indicate that the relative positions are identical—if applicable having identical Gray code values—the dose information calculated from the measurement of the motion sensing unit need not be corrected. If the comparison of the positional information yields that the position indicated by the stored positional information and the position derived from the measurements of the motion sensing unit are different appropriate corrective or alarm measures can be taken. The comparison may be done by the electronic control unit. If the detectable positions of the encoder component relative to the sensor arrangement together form a Gray code which is unique for every relative position which could be distinguished, the deviation between the stored position and first position determined by the motion sensing unit can be quantified. The quantification may comprise determining the number of unit setting increments which were missed. The quantification is, of course, limited by the number of uniquely identifiable sequential relative positions between the encoder component and the sensor arrangement of the motion sensing unit as will be discussed further below. Thus, the more uniquely identifiable relative positions there are between the encoder component and the motion sensing unit or the sensor arrangement thereof, the more unit setting increments which are potentially missed due to a non-detected change in relative position between the encoder component and the motion sensing unit could be accounted for.

If the motion sensing unit is activated only after the dose delivery operation has been commenced, this has the advantage that not only a missed dose of one unit setting increment can be compensated for but also a higher number of unit setting increments, e.g. two, three, four or even more unit setting increments can be accounted. Thus, dose units which are potentially dispensed or delivered during the wake-up time of the electronic system and/or the motion sensing unit can be compensated, e.g. software based. Of course, this advantage is also present, in case for some other reason the position is changed and the electronic system is not activated too late deliberately. The system or the device is expediently configured such, after the end of the previous dose delivery operation, the positional relation between the sensor arrangement and the encoder component does not change, e.g. by one or more ratchet interfaces.

The general process to implement the comparison of the first relative position sensed by the motion sensing unit with a stored value may be as follows:
The final relative position of the encoder component relative to the sensor arrangement is stored, preferably after a previous dose delivery operation has been completed. This may correspond to storing the last sensor reading. For example, for each sensor of the system it may be stored whether there is a detection region 1890 in the detection position of that sensor or a non-detection region (the region between two radiation detection regions 1890). The detection region, e.g. on account of its higher reflectivity in case optical encoding is used, may yield the higher signal as compared to the non-detection region. It is conceivable, to store this information in the memory of a real-time clock of the electronic system. In case a system with two sensors is used and an encoding component with detection regions and non-detection regions of the angular width of two unit increments regularly spaced in the circumferential direction, the storage of data indicative for the relative position of the encoder component relative to the sensors just needs 2 bits, for example because the information which needs to be stored is which region of the encoder component is arranged in the detection position relative to which sensor (this will be explained further below). The real-time clock may be a component which is already present in an existing system and may have residual memory which is not needed otherwise. However, it is also possible to have a dedicated memory for storing the positional information or use memory within the electronic control unit or another unit of the system. Using the real-time clock may be particularly efficient as regards an optimized power consumption. The clock may be needed in the system anyway, e.g. to provide time stamps for the signals of the motion sensing unit and/or a time stamp for dose (history) data. Also, the clock may have a power consumption which is smaller than the one of the electronic control unit, even if the control unit were used temporarily only for storing the second data up to the next dose delivery operation. Thus, using the real-time clock may have a particular advantage.
When the electronic control unit and/or the motion sensing unit is activated or switched to a state of higher power consumption, the electronic control unit may retrieve the stored positional information from the memory. This is expediently done after the dose delivery operation which is to be sensed by the motion sensing unit has been completed, e.g. as then the electronic control unit is sooner available to control or trigger or actuate the motion sensing unit.
The electronic system, e.g. the control unit or sensor arrangement, may calculate the dose information, e.g. the number of unit setting increments dispensed in the dose delivery operation, based on signals generated by the sensor(s), e.g. based on edges of detection regions passing the sensors or the associated detection positions. Here, the first measured data on the first relative position of the encoder component relative to the sensor arrangement may be compared with the stored value, preferably at the end of the dose delivery operation, as, again this guarantees that all resources are available for assessing the dose information from the currently happening operation as soon and as quickly as possible. In case differences are found, the difference may be quantified and added to the dose information determined via the motion sensing unit.

The system may be configured such that the time between the generation of the use signal to activate the electronic control unit and/or the motion sensing unit and the first data signal generated via the motion sensing unit—the first data signal may comprise signals from a plurality of sensors—is used to calculate the number of unit setting increments which have been missed by the motion sensing unit. This approach may work, as the start-up time or wake-up time until the motion sensing unit becomes operational may be constant but the number of unit setting increments which is missed may depend on the acceleration and/or the rotation speed of the encoder component (interface member 1880 or first member 1780) during that time. Thus, from the data retrieved from the motion sensing unit, the rotation speed can be estimated and due to the estimated rotation speed the number of missed unit setting increments can be estimated. This approach may not require a Gray code which is formed by the encoder component and the sensor arrangement but, of course, also works with such a code. Alternatively or additionally the Gray code may be used to quantify the angular distance which has been traveled before the motion sensing unit has become operative.

From the following considerations it will become apparent how uniquely identifiable relative positions (e.g. forming a Gray code) can be established by way of systems using sensors and encoder components with detection regions. The table below shows an example for two sensors S1 and S2. The sensors may be arranged out of phase relative to the detection regions as shown in an exemplary arrangement in FIG. 10. In the table below “D” designates that the sensor has a detection region in its detection position and “N” designates that the sensor has a non-detection region in its detection position, e.g. the sensor indicates less light is incident on it than when a detection region arranged in that position. Positions P1 through P4 are sequentially arranged in the rotation direction. Two adjacent positions may be separated by one unit setting increment.

| | | | | | | |
|---|---|---|---|---|---|---|
| Sensor | Position | P1 | P2 | P3 | P4 | P1 |

| | | | | | |
|---|---|---|---|---|---|
| S1 | D | D | N | N | D |
| S2 | D | N | N | D | D |

After P4 the next position may, again, be P1. Thus, via this configuration, a difference or offset up to three positions or unit setting increments can be compensated. For example, if the stored data indicates position P1 and the first data sensed by the motion sensing unit indicates P4, the difference is three positions or unit setting increments.

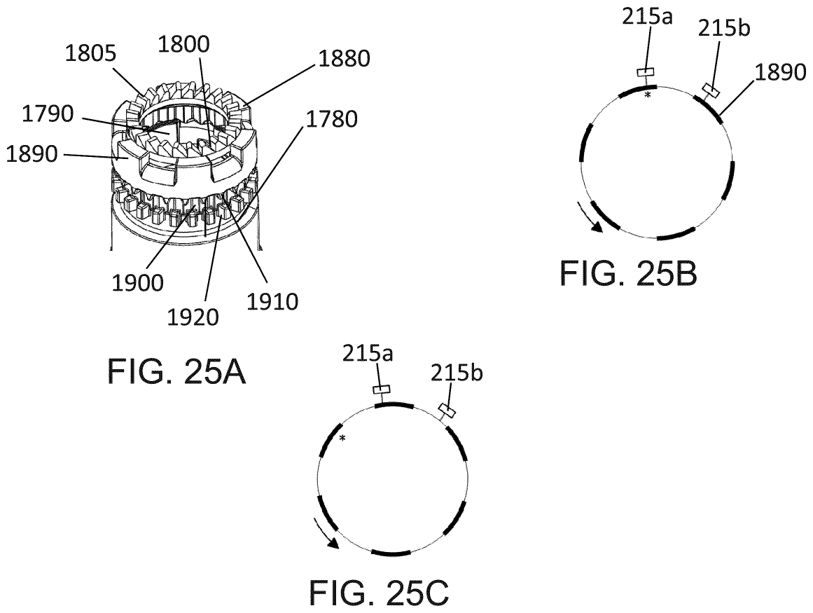

FIGS. 25A to 25C illustrate an exemplary embodiment where the sensors 215a and 215b of the FIG. 10 embodiment are used together with the encoder component 1880 shown in FIG. 24 (which is the same as shown in FIG. 25A; other encoders or other encoder/sensor combinations could be used as well, of course). The angular extension of the detection regions 1890 and/or the angular distance between the adjacent detection regions 1890 may be greater than one setting increment or one use signal generation increment, e.g. a whole-number multiple of the respective increment. The respective increment may, again, be 15°. For example, the angular extension may correspond to or be defined by the angle corresponding to a rotation by two unit setting increments or by two use signal generation increments. For example, when the member 1780 rotates relative to the housing (not explicitly shown) by an angle defined by the angular extension of the detection region 1890 in a dose delivery operation this may correspond to two dose units of drug having been dispensed from the device or a piston rod having been moved by a distance corresponding to two dose units. In other words, the angular extension and/or the angular pitch of or distance between two detection regions 1890 may be greater than the unit setting increment and/or the use signal generation increment. In the depicted embodiment, the angular extension of the respective detection region and the angular distance between two adjacent detection regions corresponds to the angle corresponding to two increments, which corresponds to an angle of 30°. If the sensors such as the sensors 215a and 215b discussed above are arranged out of phase relative to the detection regions 1890 as has been discussed further above in conjunction with FIG. 10, for example, this can be utilized in conjunction with the signal generation sequence in the sensors to compensate for an offset between the first sensed position and the stored position of one or more unit setting increments. That is to say, this arrangement may be used to compensate for unit increments which the first member 1780 has rotated relative to the second member 1790 before the rotation can be sensed or measured by the motion sensing unit 1100 which comprises the sensor arrangement with sensors 215a and 215b. In the depicted embodiment, the sensors or the locations onto which the sensors are directed, i.e. the detection positions, are separated by an odd number of (unit setting) increments. The pitch of the detection regions (their angular separation) and/or their angular width may be an even number of (unit setting) increments. It will be appreciated that “even” and “odd” may also be switched with respect to sensors and detection regions or their separation.

The electronic system 1000 may comprise a memory as has been discussed further above. Expediently, the memory is configured to store data indicative for the relative position between the detection regions 1890 and the sensors 215a and 215b, e.g. after a previous dose delivery operation has been completed and/or before the very first dose delivery operation is commenced (the very first positional data may be stored in the memory by the manufacturer, e.g. during a priming step).

In FIG. 25B the state before the dose delivery operation is commenced is depicted. This may be the initial arrangement of the sensors relative to the detection regions 1890 which is stored in the memory. In this exemplary arrangement, the first sensor 215a, with respect to the intended rotation direction during dose delivery which is indicated by the arrow, is arranged at a location closer to an end of the detection region 1890, whereas the second sensor 215b is arranged at a location closer to the start of the detection region 1890. During rotation away from the initial position depicted in FIG. 25B the use signal is generated as was discussed further above, preferably before the rotation by one use signal generation increment has been completed. Thus, in the situation depicted in FIG. 25B, the motion sensing unit, particularly, the sensors 215a, 215b may not yet be operable to detect or sense movement. The situation in FIG. 25C illustrates the situation which is detected by the sensors 215a and 215b once the motion sensing unit has been woken. As will be appreciated, the situation is different from the initial situation in that the respective sensors are arranged at locations relative to the detection regions which are different. That is to say, for example, sensor 215a is arranged close to the start of a detection region 1890 and sensor 215b is arranged in the region between two adjacent detection regions close to the start of the subsequent detection region 1890. The relative rotation which has been performed is greater than one unit setting increment, in the depicted embodiment it is three increments. In view of the unique arrangement of the two sensors relative to the detection regions, which may have a respective angular distance and an angular extension which is determined by the angle corresponding to a multiple of one increment, e.g. two increments, and the unique sequence of sensor signals indication for detection regions and non-detection regions (see the table further above), there is a plurality of, e.g. four, different distinguishable relative rotational positions, each of these positions being unique. Depending on the sensor signals and/or their sequence and, if applicable, the known rotation direction, which is preferably unidirectional only, the first positon during the dose delivery operation can be determined. This position may be compared, e.g. in the electronic control unit before or after the delivery operation has been completed, with the stored positional information on the initial relative position. From the difference it can be gathered how many unit increments have been missed before the motion sensing unit has been activated. In the present example, the motion sensing unit has been switched on after three units have already been dispensed.

Moreover, it can be advantageous to store more than one position-related data in the memory, e.g. four positions may be stored, such as every unique position. Expediently the four sequential unique positions up to the end position. If this is compared with the positional data during the dose delivery operation, the rotation direction may be validated as well and/or the correct operation of the sensor arrangement may be validated as the sequences in which the sensors should generate their signals is known.

The proposed approach assumes that the wake-up procedure (switching from the first state to the second state) of the motion sensing unit has been completed before the sensors indicate a relative position of the encoder relative to the sensor arrangement which is identical to the original one in FIG. 25B. Usually, the wake-up is completed pretty fast such that compensating an additional potential offset by using the uniquely identifiable relative positions between the sensor arrangement and the encoder component is sufficient, especially, if the number of uniquely identifiable positions, which are preferably separated by one unit setting increment, is greater than or equal to four. Of course, there is still room to compensate an even higher number of unit setting increments which could potentially be missed before the motion sensing unit is operative. For doing so, the angular extension of the detection regions 1890 may be further increased along with the number of sensors, for example. Moreover, this concept is not restricted to optoelectronic sensing but can be realized with a large variety of different sensor technologies. Also, it may be able for one sensor to distinguish not only between two states (e.g. light or dark), but a higher number of states, e.g. three or more.

The embodiment sketched in the following provides six unique positions (P1 to P6) using three sensors (S1 to S3) and two distinguishable states (N, D), where the sensors are preferably out of phase relative to the encoder component.

| | | | | | | | |
|---|---|---|---|---|---|---|---|
| Sensor | Position | P1 | P2 | P3 | P4 | P5 | P6 |

| | | | | | | |
|---|---|---|---|---|---|---|
| S1 | D | D | D | N | N | N |
| S2 | D | D | N | N | N | D |
| S3 | D | N | N | N | D | D |

The table below gives an overview over a system having, e.g. eight, uniquely identifiable sequential positions P1 to P8 along the rotation direction, where sensors S1 to S3 are used and the notation is equivalent to the one in the previous table(s). In this embodiment, different encoder components are applied for different sensors. Sensor S1 detects the position of a first encoder component relative to S1 and Sensors S2 and S3 may detect the relative position of a second encoder component relative to S2 and S3. The encoder components may differ in the sequence of the regions sensed by the respective sensor. For example, the sequence of detection regions (D) and non-detection regions (N) may be different in the first and second encoder components as will be apparent from the table below. The two encoder components are expediently rotationally locked relative to one another. Sensors S2 and S3 are out of phase relative to the second encoder component.

| | | | | | | | | | | |
|---|---|---|---|---|---|---|---|---|---|---|
| Sensor | Position | P1 | P2 | P3 | P4 | P5 | P6 | P7 | P8 | P1 |

| | | | | | | | | | |
|---|---|---|---|---|---|---|---|---|---|
| S1 | D | D | D | D | N | N | N | N | D |
| S2 | D | D | N | N | D | D | N | N | D |
| S3 | D | N | N | D | D | N | N | D | D |

Aside from having an arrangement of sensor(s) and encoder component(s) which defines two different states like “light” (a detection region D is in front of the sensor) and “dark” (a non-detection region is in front of the sensor) there is also the option of having a sensor arrangement with one or more sensors and one or more encoder components, which is configured to distinguish between more than two states. For example, If there are two sensors S1 and S2 which can distinguish between three different states, X, Y, and Z, nine uniquely identifiable relative angular positions are provided as will become apparent from the table below using two encoder components, i.e. one for each sensor.

| | | | | | | | | | | |
|---|---|---|---|---|---|---|---|---|---|---|
| Sensor | Position | P1 | P2 | P3 | P4 | P5 | P6 | P7 | P8 | P1 |

| | | | | | | | | | |
|---|---|---|---|---|---|---|---|---|---|
| S1 | X | X | X | Y | Y | Y | Z | Z | Z |
| S2 | X | Y | Z | X | Y | Z | X | Y | Z |

X, Y, and Z may represent different colors, grey scales, heights, etc.

We note that it is not necessary—although it is advantageous—to have a sequence of more than two unique positions identifiable via the signals of the motion sensing unit. A system which can discriminate between two positions may be sufficient already. This system may be a binary system. If it is known that the motion sensing unit is activated via the use signal only after the relative rotation between the members of the dose setting and drive mechanism has commenced, via the first signal it can be judged whether one unit increment or two unit increments have been missed. If one unit increment was missed the relative positional information determined via the signal of the sensing unit is different from the stored one and if two unit increments have been missed, the relative positional information determined via the signal of the sensing unit is identical to the stored one.

It should be noted that the proposed concept of checking whether there was a positional change before the motion sensing unit became operative and, potentially, compensating for unit setting increments of relative rotation which have been missed works regardless of whether the detection regions are part of the use signal generation interface member of the first member or an additional member (encoder component). Therefore, the integration of the detection regions into the use signal generation interface member is advantageous but not essential for this functionality. Consequently, the integration of detection regions into the use signal generation interface member is independent from compensating for a movement which occurred prior to the switching on of the motion sensing unit.

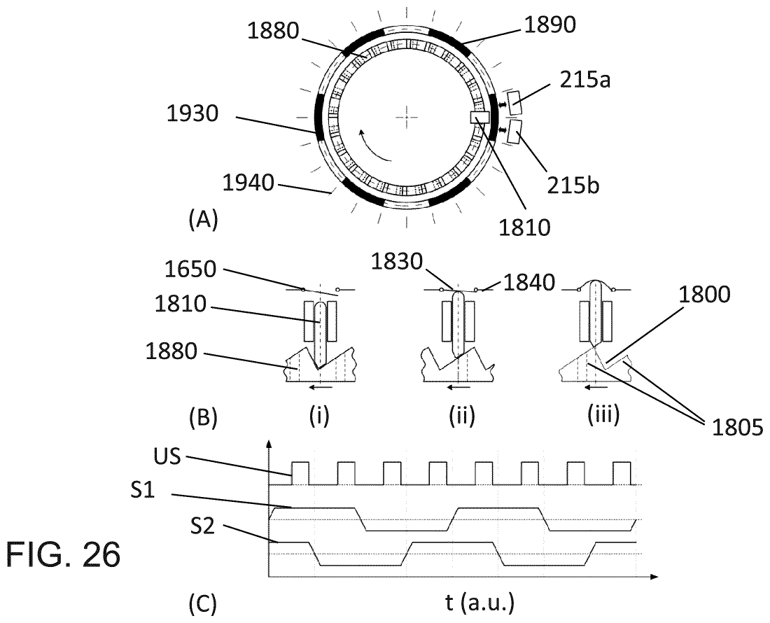

FIG. 26 illustrates another embodiment of an electronic system on the basis of three different representations. This embodiment is very similar to the embodiment described in conjunction with FIGS. 25A through 25C. As opposed to the previously discussed embodiments, FIG. 26 includes more details on the signal generation of the sensors 215a and 215b.

Representation A basically illustrates the same situation as has been described above with reference to FIGS. 25B and 25C. Specifically, representation A illustrates the use signal generation interface member 1880 where the ratchet teeth and ratchet pockets are illustrated but not labeled in this representation. Contrary to the previous representations, it also shows the switching feature 1810. The detection regions 1890 are arranged on an encoder body 1930. The encoder body 1930 may be integrated into the use signal generation interface member 1880, connected to that member or be separate from that member. The radially oriented lines 1940 which are circumferentially disposed illustrate the angles corresponding to one unit setting setting/use signal generation increment. One increment corresponds to the angle defined between two adjacent lines 1940, i.e. 15° in this embodiment. The angular extension of the detection regions 1890 and the regions between the detection regions is given by the angle corresponding to two increments, whereas two stable positions of the switching feature 1810 relative to the use signal generation interface member 1880 are separated by an angle corresponding to one increment. The sensors 215a and 215b are again out of phase but both are arranged besides one another, e.g. with detection positions which are adjacent in the angular direction. The distance between the locations onto which the sensors and/or the optoelectronic radiation source are directed in order to emit radiation onto the location and/or receive radiation reflected from the location may be the angle corresponding to one unit increment. In other words, increment-wise the sensors may be adjacent relative to one another.

Representation B illustrates the use signal generation during the rotation of the use signal generation interface member 1880 relative to the switching feature 1810. Here, a delivery sensor or switch 1650 is symbolized by a switch which may have the contact features 1830 and 1840. When the rotation starts, the system is in situation (i). Thereafter due to the relative rotational movement between the switching feature 1810 and the member 1880 and an account of the ratchet teeth 1805 having an inclined surface the switching feature 1810 is displaced, e.g. towards the switch 1650, in order to close the switch by bringing the contacts 1830 and 1840 in mechanical contact to generate the use signal. In situation (ii) the switch has been closed and the use signal is being generated. As previously described, the switch 1650 is expediently designed such that it closes before the switching feature 1810 has reached its end position relative to member 1880. Thus, from situation (ii) the switching feature is displaced further and the contacts 1830 and 1840 are kept in contact such that the use signal is still generated. Situation (iii) illustrates the situation shortly before the switching feature 1810 engages the subsequent ratchet pocket 1800, which corresponds to the end of use signal generation, e.g. by the contact features 1830 and 1840 disengaging.

Representation C illustrates schematically various electrical signals over time t during the relative rotation of the encoder body 1930 relative to the sensors. At the top the use signal US is shown. S1 is the signal generated via the sensor 215a and S2 is the signal generated while the sensor 215b. As will be appreciated, when comparing the sensor signals S1 and S2 their temporal sequence is different due to the sensors being arranged out of phase and, consequently, it can be gauged by how much the first member 1780 has rotated before the sensors became active if the situation after the latest dose delivery operation or in the “as-delivered state” is appropriately stored as has been discussed above.

The proposed concepts may yield a unit increment or even sub-unit increment accuracy in dose information determination, even if the motion sensing unit is powered-up (rendered operative or switched to the second state) after the deliver operation has been commenced already. The concepts are not only suitable to be used when the motion sensing unit is rendered operative after the delivery operation was initiated but are also suitable to check whether the first position determined via the sensing unit matches the expected position which had been stored previously, e.g. to check whether the device functions properly.

Moreover, although the proposed concepts are particularly suitable for dose delivery operations, they may, nevertheless, be applied for dose setting operations as well.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-carboxyheptadecanoyl)-des(B30) human insulin and B29-N-(w-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

The scope of protection is not limited to the examples given herein above. Any invention disclosed herein is embodied in novel characteristics or a combination of characteristics disclosed here or understandable from this disclosure even if not explicitly stated herein.

### REFERENCE NUMERALS

1 device
2 device
10 housing
12 dosage knob
11 injection button
13 dosage window
14 container or container receptacle
15 needle
16 inner needle cap
17 outer needle cap
18 cap
27 output
28 switch
29 power supply
70 dial sleeve
70a segment
70b segment
71a . . . formation
205 grip
210 injection button
210a button part
210b button part
215 sensor arrangement
215a sensor
215b sensor
500 encoder system
700 controller
800 switch
900 encoder system
1000 electronic system
1100 electronic control unit
1200 motion sensing unit
1300 use detection unit
1400 communication unit
1500 power supply
1600 user interface member
1650 switch
1780 first member
1790 second member
1800 ratchet pockets
1805 ratchet teeth
1820 guide slot
1830 first contact feature
1840 second contact feature
1850 protrusion
1860 surface
1870 ramp
1880 use signal generation interface member
1890 detection region
1900 spline feature
1910 engagement feature
1920 engagement feature
1930 encoder body
1940 line
S1 sensor signal
S2 sensor signal
US use signal
d_{c} clutch release distance

1-19. (canceled)

20. An electronic system for a drug delivery device, the electronic system comprising:
an electronic control unit configured to control an operation of the electronic system; and
an electrical motion sensing unit configured to generate one or more data signals, the one or more data signals being suitable to quantify relative rotational movement between a first member and a second member during a dose setting operation for setting a dose to be delivered by the drug delivery device and/or during a dose delivery operation for delivering the set dose,
wherein the electronic system is configured to perform a comparison operation to compare first data to second data, the first data indicating a first relative angular position of the first member and the second member, and being derivable from the one or more data signals, the second data indicating a second relative angular position of the first member and the second member.

21. The electronic system of claim 20, wherein the first data is data indicative of a detectable relative angular position between the first member and the second member which is sensed by the motion sensing unit during the dose setting operation or the dose delivery operation.

22. The electronic system of claim 20, wherein the second data is data indicative of a relative angular position between the first member and the second member at an end of a previous dose setting or dose delivery operation and/or before commencement of the dose setting operation or the dose delivery operation.

23. The electronic system of claim 20, wherein the electrical motion sensing unit is operative only during the dose delivery operation.

24. The electronic system of claim 20, wherein the electronic system is configured such that the motion sensing unit is rendered operative only after the dose setting operation or the dose delivery operation has been commenced.

25. The electronic system of claim 20, wherein the electronic system is further configured to use the comparison operation to determine whether there is an offset between (i) dose information characteristic for the dose setting operation or the dose delivery operation derived or derivable from the motion sensing unit and (ii) an actual dose information characteristic for the dose setting operation or the dose delivery operation.

26. The electronic system of claim 25, wherein the offset is a difference between stored data and the first data.

27. The electronic system of claim 20, wherein the electronic system is further configured to use the comparison operation to detect an offset of more than one unit setting increment between the first and the second relative angular position.

28. The electronic system of claim 20, further comprising an electrical use detection unit operatively connected to the electronic control unit, the electrical use detection unit being configured to generate a use signal indicating that a user has commenced or is about to commence the dose setting operation or the dose delivery operation,
wherein the electronic system is configured such that in response to the use signal, the electronic control unit switches the motion sensing unit from a first state, in which the motion sensing unit is not operative, into a second state, in which the motion sensing unit is operative.

29. The electronic system of claim 28, wherein the electrical use detection unit is configured to generate the use signal in response to relative movement between the first member and the second member.

30. The electronic system of claim 20,
wherein the motion sensing unit comprises a sensor arrangement comprising one or more sensors, and
wherein the electronic system further comprises an encoder component with a plurality of detection regions that are configured to excite the one or more sensors of the sensor arrangement,
wherein each of the detection regions is suitable to assume a detection position relative to at least one respective sensor in the one or more sensors, and
wherein one of the sensor arrangement and the encoder component is associated with the first member and the other one of the sensor arrangement and the encoder component is associated with the second member.

31. The electronic system of claim 30, wherein two or more of (i) the number of sensors of the sensor arrangement, (ii) an arrangement of the sensors of the sensor arrangement relative to one another, (iii) an arrangement of the detection regions relative to the sensors of the sensor arrangement, or (iv) a configuration of the detection regions of the encoder component are adjusted to one another such that there is a number N of uniquely identifiable sequential relative angular positions between the encoder component and the sensor arrangement, wherein N is greater than or equal to 2.

32. The electronic system of claim 30, wherein an angular extension of each of the detection regions is greater than one unit setting increment.

33. The electronic system of claim 20, wherein the second data comprises data on a sequence of successive relative angular positions of the first member and the second member.

34. The electronic system of claim 20, further comprises a real-time clock comprising a memory, wherein the second data is retrieved or retrievable in or for the comparison operation from the memory.

35. The electronic system of claim 20, wherein the dose setting and drive mechanism is configured such that after a type of operation from among the dose setting operation and the dose delivery operation has been completed, a relative angular position between the first member and the second member does not change before a subsequent operation of the same type is conducted.

36. The electronic system of claim 20, wherein in response to detecting a difference in the relative position during the comparison operation, the electronic system is configured to use the difference to adjust dose information derived from relative rotational movement between the first member and the second member during the dose setting operation or dose delivery operation.

37. The electronic system of claim 20, wherein the electronic system is configured such that the comparison operation is performed during the dose delivery operation.

38. A drug delivery device comprising:
an electronic system; and
a reservoir with a drug,
wherein the electronic system comprises
an electronic control unit configured to control an operation of the electronic system, and
an electrical motion sensing unit configured to generate one or more data signals, the one or more data signals being suitable to quantify relative rotational movement between a first member and a second member during a dose setting operation for setting a dose to be delivered by the drug delivery device and/or during a dose delivery operation for delivering the set dose,
wherein the electronic system is configured to perform a comparison operation to compare first data to second data, the first data being indicative of a first relative angular position of the first member and the second member, and being derivable from the one or more data signals, the second data being indicative of a second relative angular position of the first member and the second member.

39. A drug delivery device comprising:
an electronic system; and
a reservoir retainer for retaining a reservoir with a drug in the drug delivery device,
wherein the electronic system comprises
an electronic control unit configured to control an operation of the electronic system, and
an electrical motion sensing unit configured to generate one or more data signals, the one or more data signals being suitable to quantify relative rotational movement between a first member and a second member during a dose setting operation for setting a dose to be delivered by the drug delivery device and/or during a dose delivery operation for delivering the set dose,
wherein the electronic system is configured to perform a comparison operation to compare first data to second data, the first data being indicative of a first relative angular position of the first member and the second member, and being derivable from the one or more data signals, the second data being indicative of a second relative angular position of the first member and the second member.
